(19) United States
(12) Patent Application Publication
(10) Pub. No.: US 2023/0398216 A1
(43) Pub. Date: Dec. 14, 2023

(54) CHIMERIC ACTIVATION RECEPTORS

(71) Applicant: Lyell Immunopharma, Inc., South San Francisco, CA (US)

(72) Inventors: Queenie M. VONG, South San Francisco, CA (US); Blythe D. SATHER, South San Francisco, CA (US)

(73) Assignee: Lyell Immunopharma, Inc., South San Francisco, CA (US)

(21) Appl. No.: 18/250,156

(22) PCT Filed: Oct. 22, 2021
(86) PCT No.: PCT/US2021/056303

§ 371 Date: Apr. 21, 2023

Related U.S. Application Data
(60) Provisional application No. 63/104,419, filed on Oct. 22, 2020.

(51) Int. Cl. A61K 39/00; C12N 15/86; C07K 14/725; C07K 14/71; C07K 14/705; C07K 16/28; C07K 16/32

(52) U.S. Cl. CPC A61K 39/4631; C12N 15/86; C07K 14/7051; C07K 14/71; C07K 14/70507; C07K 16/2803; C07K 16/32; C07K 14/70521; A61K 39/4612; A61K 39/4611; A61K 39/4613; A61K 39/464462; A61K 39/464474; C12N 2740/15043; C07K 2319/03

(57) ABSTRACT

The preset disclosure provides chimeric activation receptors comprising (i) a TGFβ-binding domain and (ii) a CD2 costimulatory domain. In some aspects, the TGFβ-binding domain comprises an extracellular domain of a TGFβ receptor. Other aspects of the disclosure are directed to nucleic acid molecules encoding a chimeric activation receptor, cells comprising the chimeric activation receptor and/or a nucleic acid molecule encoding the same, and methods of use thereof in the treatment of a disease or condition (e.g., a tumor) in a subject in need thereof.

### CROSS-REFERENCE TO RELATED APPLICATIONS

This application claims priority benefit of U.S. Provisional Application No. 63/104,419, filed Oct. 22, 2020, which is herein incorporated by reference in its entirety.

### REFERENCE TO SEQUENCE LISTING SUBMITTED ELECTRONICALLY VIA EFS-WEB

The content of the electronically submitted sequence listing (Name: 4385_040PC01_Seqlisting_ST25.txt, Size: 19,807 bytes; and Date of Creation: Oct. 22, 2021) submitted in this application is incorporated herein by reference in its entirety.

### FIELD

The present disclosure relates to chimeric activation receptors, nucleic acid molecules encoding the same, and cells (e.g., immune cells) comprising the same. The cells disclosed herein can be used in various cell therapies, including but not limited to chimeric antigen receptor (CAR) T cell therapy and TCR T cell therapy, including neoantigen directed-T cell therapies.

### BACKGROUND

Cancer immunotherapy relies on harnessing T cells—the immune system's primary killers of infected and diseased cells—to attack and kill tumor cells. However, the ability of immune cells to target and kill tumor cells is dampened by the presence of various inhibitors of the immune response that are present within the tumor microenvironment. Among these inhibitors is transforming growth factor β (TGFβ), which acts to suppress the immune response through TGFβ receptors expressed by T cells.

Conventional means of combating TGFβ inhibition of T cells in the tumor microenvironment include administration of TGFβ inhibitors or the use of modified T cells expressing a TGFβR dominant negative (DN). More broad techniques, such as systemic inhibitors, can have effects beyond just the tumor microenvironment. Given the pleiotropic effect of TGFβ, one potential concern of systemic therapy is the development of autoimmune toxicities in humans. The systemic blockade of TGFβ might affect cytokine's homeostatic function in other tissues outside the immune system. TGFβ signaling can be blocked by expressing a dominant-negative TGFBRII (TGFβRII-DNR), which is truncated and lacks the intracellular domain necessary for downstream signaling in order to reduce the inhibitory effect of TGFβ. Expression of the TGFβRII-DNR enhances antitumor immunity, however, if expressed at the wrong time or by the wrong promoter during T cell development (in mouse models) it can lead to autoimmunity or lymphoproliferative disorder. Safety and efficacy of the TGFβRII-DNR in Epstein-Barr virus (EBV)-specific T cells for lymphoma has been evaluated in clinical trial (NCT00368082) and when combined with a PSMA CAR (NCT04249947). Neither trial reported overt toxicity related to the expression of the TGFbRII-DN but enhancement of efficacy was unclear.

Thus, there remains a need in the art to further enhance the immune response in the tumor microenvironment.

### BRIEF SUMMARY

Certain aspects of the present disclosure are directed to an immune cell comprising a chimeric activation receptor, wherein the chimeric activation receptor comprises (i) a transforming growth factor β (TGFβ)-binding domain; (ii) a transmembrane domain; (iii) and a CD2 costimulatory domain; wherein the immune cell expresses an endogenous TGFβRI and/or TGFβRII.

In some aspects, the immune cell is selected from a T cell, a B cell, a regulatory T cell (Treg), a natural killer (NK) cell, a natural killer T (NKT) cell, a stem cell, an induced pluripotent stem cell, and any combination thereof. In some aspects, the chimeric activation receptor is capable of competing with binding of an endogenous TGFβRI and/or an endogenous TGFβRII to TGFb. In some aspects, the chimeric activation receptor is capable of forming a heterotetradimer with an endogenous TGFβRI and/or an endogenous TGFβRII.

In some aspects, the TGFβ-binding domain is an extracellular domain of TGFβRII.

In some aspects, upon interaction of the chimeric activation receptor with TGFβ, the immune cell produces one or more cytokines at a higher level than a cell expressing a TGFβRII-binding domain fused to a CD28 costimulatory domain upon interaction with TGFβ. In some aspects, the one or more cytokines are selected from IL2 and IFNγ.

In some aspects, upon the interaction with TGFβ, the immune cell expresses IL2 at a level that is at least about 125%, at least about 150%, at least about 200%, at least about 250%, at least about 300%, at least about 350%, at least about 400%, at least about 450%, at least about 500% the expression of IL2 by a cell expressing a TGFβRII-binding domain fused to a CD28 costimulatory domain upon binding to TGFβ. In some aspects, upon the interaction with TGFβ, the immune cell expresses IFNγ at a level that is at least about 125%, at least about 150%, at least about 200%, at least about 250%, at least about 300%, at least about 350%, at least about 400%, at least about 450%, at least about 500% the expression of IFNγ by a cell expressing a TGFβRII-binding domain fused to a CD28 costimulatory domain upon binding to TGFβ.

In some aspects, the expression of the one or more cytokines by the immune cell is higher than a cell expressing a TGFβRII-binding domain fused to a CD28 costimulatory domain, as measured at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

In some aspects, upon the interaction with TGFβ, the immune cell is more proliferative than a cell comprising a TGFβRII-binding domain fused to a CD28 costimulatory domain upon interaction with TGFβ. In some aspects, upon interaction with TGFβ, the immune cell is at least about 25% more, at least about 50% more, at least about 75% more, at least about 100% more, at least about 125% more, at least about 150% more, at least about 175% more, at least about 200% more, at least about 250% more, at least about 300% more, at least about 350% more, at least about 400% more, at least about 450% more, at least about 500% more proliferative than a cell comprising a TGFβRII-binding domain fused to a CD28 costimulatory domain upon interaction with TGFβ. In some aspects, upon interaction with TGFβ, the immune cell is more proliferative than a cell comprising a TGFβRII-binding domain fused to a CD28 costimulatory domain, as measured at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

In some aspects, upon binding to TGFβ, the immune cell has increased cytolytic activity as compared to a cell comprising a TGFβRII-binding domain fused to a CD28 costimulatory domain upon binding to TGFβ. In some aspects, upon interaction with TGFβ, the immune cell has a cytolytic activity that is at least about 125%, at least about 150%, at least about 200%, at least about 250%, at least about 300%, at least about 350%, at least about 400%, at least about 450%, at least about 500% the cytolytic activity of a cell comprising a TGFβRII-binding domain fused to a CD28 costimulatory domain upon interaction with TGFβ. In some aspects, upon interaction with TGFβ, the immune cell has increased cytolytic activity as compared to a cell comprising a TGFβRII-binding domain fused to a CD28 costimulatory domain, as measured at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

In some aspects, the TGFβ-binding domain comprises the extracellular domain of wild-type human TGFβRII. In some aspects, the TGFβ-binding domain comprises the extracellular domain of human TGFβRII having one or more point mutation relative to wild-type human TGFβRII, which increases the binding affinity of the extracellular domain of TGFβRII to TGFβ. In some aspects, the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6. In some aspects, the TGFβ-binding domain comprises the amino acid sequence set forth in SEQ ID NO: 6.

In some aspects, the transmembrane domain comprises the transmembrane domain selected from the group consisting of wild-type human TGFβRII, a CD8 transmembrane domain, a CD2 transmembrane domain, and any combination thereof. In some aspects, the transmembrane domain comprises a CD8 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5, 8, or 9. In some aspects, the transmembrane domain comprises the amino acid sequence set forth in SEQ ID NO: 5, 8, or 9.

In some aspects, the CD2 costimulatory domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7. In some aspects, the CD2 costimulatory domain of comprises the amino acid sequence set forth in SEQ ID NO: 7.

In some aspects, the immune cell further comprises a chimeric antigen receptor and/or a TCR. In some aspects, the chimeric antigen receptor comprises an antigen-binding domain that specifically binds a molecule expressed by a tumor cell. In some aspects, the chimeric antigen receptor comprises an antigen-binding domain that specifically binds an antigen selected from the group consisting of AFP (alpha-fetoprotein), αvβ6 or another integrin, BCMA, Braf, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRCSD (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MC SP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof.

In some aspects, the chimeric antigen receptor comprises an antigen-binding domain that specifically binds ROR1. In some aspects, the chimeric antigen receptor comprises an antigen-binding domain that specifically binds GPC2. In some aspects, the chimeric antigen receptor comprises a costimulatory domain selected from a costimulatory domain from interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, and any combination thereof. In some aspects, the chimeric antigen receptor comprises a 4-1BB/CD137 costimulatory domain.

In some aspects, the TCR specifically binds a tumor antigen. In some aspects, the TCR specifically binds an antigen selected from the group consisting of AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3E, CD4, CD5, CD7, the extracellular portion of the APRIL protein, and any combinations thereof.

Certain aspects of the present disclosure are directed to a nucleic acid encoding a chimeric activation receptor disclosed herein.

Certain aspects of the present disclosure are directed to a nucleic acid encoding a chimeric activation receptor comprising (i) a transforming growth factor β (TGFβ)-binding domain; (ii) a transmembrane domain; (iii) and a CD2 costimulatory domain.

In some aspects, the TGFβ-binding domain is an extracellular domain of TGFβRII. In some aspects, the TGFβ-binding domain comprises the extracellular domain of wild-type human TGFβRII. In some aspects, the TGFβ-binding domain comprises the extracellular domain of human TGFβRII having one or more point mutation relative to wild-type human TGFβRII, which increases the binding affinity of the extracellular domain of TGFβRII to TGFβ. In some aspects, the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6. In some aspects, the TGFβ-binding domain comprises the amino acid sequence set forth in SEQ ID NO: 6.

In some aspects, the transmembrane domain comprises the transmembrane domain of wild-type human TGFβRII. In some aspects, the transmembrane domain comprises a CD8 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5, 8, or 9. In some aspects, the transmembrane domain comprises the amino acid sequence set forth in SEQ ID NO: 5, 8 or 9.

In some aspects, the CD2 costimulatory domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7. In some aspects, the CD2 costimulatory domain of comprises the amino acid sequence set forth in SEQ ID NO: 7.

In some aspects, the nucleic acid further encodes a chimeric antigen receptor. In some aspects, the chimeric antigen receptor comprises an antigen-binding moiety that specifically binds a target molecule expressed by a tumor cell. In some aspects, the antigen-binding moiety comprises a fragment of an antibody. In some aspects, the antigen-binding moiety comprises an scFv, a nanobody, a VHH, an Fab, a DARPin, a vNAR, or an affibody.

In some aspects, the antigen-binding moiety specifically binds an antigen selected from the group consisting of AFP (alpha-fetoprotein), αvβ6 or another integrin, BCMA, Braf, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRCSD (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MC SP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof. In some aspects, the antigen-binding moiety specifically binds GPC2. In some aspects, the antigen-binding moiety specifically binds ROR1. In some aspects, the nucleic acid further encodes a linker between the chimeric antigen receptor and the chimeric signaling receptor.

In some aspects, the nucleic acid further encodes a TCR. In some aspects, the TCR specifically binds a tumor antigen. In some aspects, the TCR specifically binds an antigen selected from the group consisting of AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin Bl, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

In some aspects, the nucleic acid further encodes a linker between the TCR and the chimeric signaling receptor. In some aspects, the linker is a cleavable linker. In some aspects, the linker is selected from a P2A linker, a T2A linker, an F2A linker, an E2A linker, a furin cleavage site, or any combination thereof. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 11. In some aspects, the linker comprises the amino acid sequence set forth in SEQ ID NO: 11. In some aspects, the nucleic acid molecule comprises an IRES in between the portion of the nucleic acid encoding the chimeric antigen receptor and the portion of the nucleic acid encoding the chimeric activation receptor.

Certain aspects of the present disclosure are directed to an expression vector comprising a nucleic acid disclosed herein operably linked to a regulatory sequence. In some aspects, the expression vector is a lentiviral vector, a retroviral vector, a bacterial vector, a DNA plasmid, a dsDNA fragment, an ssDNA fragment, or any combination thereof.

Certain aspects of the present disclosure are directed to a chimeric activation receptor comprising (i) a transforming growth factor β (TGFβ)-binding domain; (ii) a transmembrane domain; (iii) and a CD2 costimulatory domain. In some aspects, the TGFβ-binding domain is an extracellular domain of TGFβRII. In some aspects, the TGFβ-binding domain comprises the extracellular domain of wild-type human TGFβRII. In some aspects, the TGFβ-binding domain comprises the extracellular domain of human TGFβRII having one or more point mutation relative to wild-type human TGFβRII, which increases the binding affinity of the extracellular domain of TGFβRII to TGFβ. In some aspects, the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6. In some aspects, the TGFβ-binding domain comprises the amino acid sequence set forth in SEQ ID NO: 6.

In some aspects, the transmembrane domain comprises the transmembrane domain of wild-type human TGFβRII, CD2, CD8, or any combination thereof. In some aspects, the transmembrane domain comprises a CD8 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5, 8, or 9. In some aspects, the transmembrane domain comprises the amino acid sequence set forth in SEQ ID NO: 5, 8, or 9.

In some aspects, the CD2 costimulatory domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7. In some aspects, the CD2 costimulatory domain comprises the amino acid sequence set forth in SEQ ID NO: 7.

Certain aspects of the present disclosure are directed to a chimeric activation receptor, encoded by a nucleic acid disclosed herein.

Certain aspects of the present disclosure are directed to a pharmaceutical composition comprising an immune cell disclosed herein, a nucleic acid disclosed herein, a vector disclosed herein, or a chimeric activation receptor disclosed herein.

Certain aspects of the present disclosure are directed to a method of preparing a cell expressing a chimeric activation receptor comprising transfecting a cell with a nucleic acid disclosed herein.

Certain aspects of the present disclosure are directed to a method of preparing a cell expressing a chimeric activation receptor comprising transducing a cell with a viral vector comprising a nucleic acid disclosed herein.

Certain aspects of the present disclosure are directed to a method of converting an endogenous TGFβR activity to a stimulatory signaling in a cell comprising transfecting the immune cell with a nucleic acid disclosed herein.

Certain aspects of the present disclosure are directed to a method of modulating TGFβ activity in a tumor microenvironment comprising administering an immune cell disclosed herein.

Certain aspects of the present disclosure are directed to a method of treating a tumor in a subject in need thereof, comprising administering to the subject an immune cell disclosed herein. In some aspects, the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof. In some aspects, the tumor is a solid tumor. In some aspects, the tumor microenvironment comprise one or more cells that express TGFβ. In some aspects, a tumor cell expresses TGFβ. In some aspects, one or more fibroblasts, MDSC-myeloid derived suppressor cells, Treg, macrophages, or any combination thereof in the tumor microenvironment express TGFβ.

### BRIEF DESCRIPTION OF THE DRAWINGS/FIGURES

FIG. 1 shows a schematic diagram of the transforming growth factor-β (TGF-β) signaling pathway and a representation of the dominant-negative TGF-β type II receptor lacking the cytoplasmic domain necessary for TGF-β signaling. Adding an additional costimulatory signaling domain uniquely linked to the truncated TGFβ receptor 2 (TGFβR2) converts an immunosuppressive TGFβ signal to a stimulatory signal to alter T cell proliferation/persistence/anti-tumor activities.

FIG. 2 is an illustrative diagram of various TGFβR2-DNR chimeric receptor constructs that were designed. “SP1” refers to a spacer. Any spacer known in the art, e.g., one or more amino acids, can be used in the constructs disclosed herein.

FIGS. 3A-3C are graphical representations of fold expansion of human ROR1 CAR+ T cells from three different donors cocultured with H1975 target cells to evaluate the anti-tumor activity with the TGFβR2-DNR chimeric receptor in the presence or absence of TGFβ-enriched environment.

FIGS. 4A-4F are graphical representations of IFNg production by human ROR1 CAR+ T cells from three different donors cocultured with H1975 cells (FIGS. 4A-4C) or A549 cells (FIGS. 4D-4E) ROR1+ target cells as.

FIGS. 5A-5F are graphical representations of IL-2 production by human ROR1 CAR+ T cells from three different donors cocultured with H1975 cells (FIGS. 5A-5C) or A549 cells (FIGS. 5D-5E) ROR1+ target cells as.

FIGS. 6A-6C are graphical representations of results from a sequential killing assays of ROR1 CAR+ T cells cocultured with H1975 ROR1+ target cells. The results showed that anti-ROR1 CAR T cells co-expressing TGFβR2-DNR, TGFβR2-DNR-CD2 and TGFβR2-DNR-OX40 were able to sustain potent target lytic activities over several rounds of target exposure.

FIGS. 7A-7C are graphical representations of results from a serial stimulation assay (of ROR1 CAR+ T cells restimulated with H1975 ROR1+ target cells in the presence or absence of TGFβ. ROR1 CAR+ T cell numbers were measured and reset to 1:1 E to T ratio every 7 days. Data was obtained from three different donors.

FIGS. 8A-8F are bar graphs, illustrating the fold expansion of ROR1 CAR+ T cells subjected to serial stimulation by H1975 ROR1+ target cells in the presence or absence of TGFβ, using ROR1 CAR+ T cells from three different donors, at day 14 (FIGS. 8A-8C) and day 21 (FIGS. 8D-8F).

FIGS. 9A-9C are bar graphs showing the production of IL-2 twenty-four hours after each round of restimulation (specified SS1, SS2 and SS3 for serial stimulation 1-3). Data are shown from culture with or without the addition of exogenous TGFb, as measured from the supernatant of the culture by MSD assay.

FIG. 10 is a diagram illustrating various TGFβR2-DNR chimeric receptor constructs that were designed to evaluate the effect of CD8 and CD2 transmembrane domains link with CD2 intracellular domain comparing against CD8 transmembrane domain link with CD28 intracellular domain. “SP1” refers to a spacer.

FIGS. 11A-11B are graphical representations of results from a serial stimulation assay of ROR1 CAR+ T cells restimulated with H1975 ROR1+ target cells in the presence or absence of TGFβ. ROR1 CAR+ T cell numbers were measured and reset to 1:1 E to T ratio every 7 days. Data was obtained from two different donors.

FIGS. 12A-12B are bar graphs showing the production of IL-2 twenty-four hours after each round of restimulation (specified SS1, SS2, SS3, SS4 for serial stimulation 1-4). Data are shown from culture with or without the addition of exogenous TGFb, as measured from the supernatant of the culture by MSD assay. Data were obtained from two different donors.

FIGS. 13A-13F are bar graphs showing target specific proliferation of ROR1 CAR+ cell against A549 (ROR1+ cell line) or A549-ROR1 knockout cells in the presence or absence of TGFb at day 7 (FIGS. 13A-13C) or day 14 (FIGS. 13D-13F).

### DETAILED DESCRIPTION

One of the most promising advancements in fighting cancer has been the development of various forms of immunotherapy. By enhancing an immune response in a subject, these therapies allow a subject's own immune system to target and kill tumor cells. However, the tumor microenvironment often has multiple elements that inhibit an immune response against a tumor. For example, transforming growth factor β (TGFβ) is expressed by multiple cell types in and around the tumor, including fibroblasts, MDSC-myeloid derived suppressor cells, regulatory T cells, and macrophages. This creates a tumor microenvironment that has a relatively high local concentration of TGFβ. Binding of TGFβ to TGFβ receptors expressed on T cells results in expression of immunosuppressive signals in the T cell, dampening the immune response against the tumor. Conventional means of blocking this TGFβ-induced inhibition of the immune response include using a TGFβ inhibitor or blocking antibody, which can have detrimental effects beyond the tumor microenvironment due to the nonspecific nature of these approaches. Alternatively, co-expression of a TGFβR dominant negative receptor can act as a “sink” for resident TGFb, competing with endogenous TGFbRII and preventing the inhibitory signals specifically on the cell product. The chimeric activation receptors described herein go beyond simply blocking TGFβ-induced inhibition of the immune response and convert this inhibitory signal into a simulator of the immune response, effectively turning an inhibitor that is at a high concentration within the tumor microenvironment into an activator of the immune response.

Certain aspects of the present disclosure are directed to chimeric activation receptors comprising (i) a transforming growth factor β (TGFβ)-binding domain; (ii) a transmembrane domain; (iii) and a CD2 costimulatory domain. In some aspects, the chimeric activation receptor is capable of competing with an endogenous TGFβRI and/or an endogenous TGFβRII for binding to TGFβ. In some aspects, the TGFβ-binding domain comprises an extracellular domain of TGFβRI or a fragment thereof, wherein the fragment retains the ability to interact with TGFβ. In some aspects, the TGFβRI is a human TGFβRI. In some aspects, the TGFβ-binding domain comprises an extracellular domain of TGFβRII or a fragment thereof, wherein the fragment retains the ability to interact with TGFβ. In some aspects, the TGFβRII is a human TGFβRI.

Other aspects of the present disclosure are directed to a nucleic acid molecule encoding a chimeric activation receptor disclosed herein. In some aspects, the nucleic acid molecule further encodes a chimeric antigen receptor (CAR) and/or a T cell receptor (TCR). Other aspects of the present disclosure are directed to an immune cell comprising a chimeric activation receptor disclosed herein or a nucleic acid molecule disclosed herein. In some aspects, the immune cell further comprises a CAR and/or a TCR.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, “transforming growth factor beta” or “TGFβ” refers to a pleiotropic cytokine that is secreted by fibroblasts, epithelial cells and a range of other cell types in the tumor microenvironment, in a tissue specific manner and functions in a context-dependent fashion. There are three known mammalian family members, (TGFβ1, TGFβ2, and TGFβ3), each of which functions through the same receptor signaling systems. TGFβ is synthesized in a latent form that must be activated to allow for engagement of a tetrameric receptor complex composed of TGFβ receptors I and II (TGFβRI and TGFβRII). The production and activation of TGFβ can be mediated by distinct cellular sources, providing additional complexity to the regulation of this pleiotropic cytokine. The amino acid sequence of TGFβRI is shown in Table 1 (UniProtKB—P36897; SEQ ID NO: 1). The transmembrane region of TGFβRI is SEQ ID NO: 2, which is amino acids 127-147 of SEQ ID NO: 1. The extracellular domain of TGFβRI is SEQ ID NO: 3, which is amino acids 34-126 of SEQ ID NO: 1. The amino acid sequence of TGFβRII is shown in Table 1 (UniProtKB—P37173; SEQ ID NO: 4). The transmembrane region of TGFβRII is SEQ ID NO: 5, which is amino acids 167-187 of SEQ ID NO: 4. The extracellular domain of TGFβRII is SEQ ID NO: 6, which is amino acids 23-166 of SEQ ID NO: 4.

TABLE 1
Human TGFβ Receptor Sequences.

| | |
|---|---|
| Human | MEAAVAAPRPRLLLLVLAAAAAAAAALLPGATA |
| TGFβRI | LQCFCHLCTKDNFTCVTDGLCFVSVT |
| (signal | ETTDKVIHNSMCIAEIDLIPRDRPFV |
| peptide; | CAPSSKTGSVTTTYCCNQDHCNKIEL |
| extra- | PTTVKSSPGLGPVEL |
| cellular | AAVIAGPVCFVCISLMLMVYICHNRTVIHHRVP |
| domain; | NEEDPSLDRPFISEGTTLKDLIYDMTTSGSGSG |
| trans- | LPLLVQRTIARTIVLQESIGKGRFGEVWRGKWR |
| membrane | GEEVAVKIFSSREERSWFREAEIYQTVMLRHEN |
| domain) | ILGFIAADNKDNGTWTQLWLVSDYHEHGSLFDY |
| | LNRYTVTVEGMIKLALSTASGLAHLHMEIVGTQ |
| | GKPAIAHRDLKSKNILVKKNGTCCIADLGLAVR |
| | HDSATDTIDIAPNHRVGTKRYMAPEVLDDSINM |
| | KHFESFKRADIYAMGLVFWEIARRCSIGGIHED |
| | YQLPYYDLVPSDPSVEEMRKVVCEQKLRPNIPN |
| | RWQSCEALRVMAKIMRECWYANGAARLTALRIK |
| | KTLSQLSQQEGIKM |
| | (SEQ ID NO: 1) |
| Human | AAVIAGPVCFVCISLMLMVYI |
| TGFβRI | (SEQ ID NO: 2) |
| trans- | |
| membrane | |
| domain | |
| Human | LQCFCHLCTKDNFTCVTDGLCFVSVTETTDKVI |
| TGFβRI | HNSMCIAEIDLIPRDRPFVCAPSSKTG |
| extra- | SVTTTYCCNQDHCNKIELPTTVKSSPGLGPVEL |
| cellular | (SEQ ID NO: 3) |
| domain | |
| Human | MGRGLLRGLWPLHIVLWTRIAS |
| TGFβRII | TIPPHVQKSVNNDMIVTDNNGAVKFP |
| (signal | QLCKFCDVRFSTCDNQKSCMSNCSIT |
| peptide; | SICEKPQEVCVAVWRKNDENITLETV |
| extra- | CHDPKLPYHDFILEDAASPKCIMKEK |
| cellular | KKPGETFFMCSCSSDECNDNIIFSEE |
| domain; | YNTSNPDLLLVIFQ |
| trans- | VTGISLLPPLGVAISVIIIFYCYRVNRQQKLSS |
| membrane | TWETGKTRKLMEFSEHCAIILEDDRSDISSTCA |
| domain) | NNINHNTELLPIELDTLVGKGRFAEVYKAKLKQ |
| | NTSEQFETVAVKIFPYEEYASWKTEKDIFSDIN |
| | LKHENILQFLTAEERKTELGKQYWLITAFHAKG |
| | NLQEYLTRHVISWEDLRKLGSSLARGIAHLHSD |
| | HTPCGRPKMPIVHRDLKSSNILVKNDLTCCLCD |
| | FGLSLRLDPTLSVDDLANSGQVGTARYMAPEVL |
| | ESRMNLENVESFKQTDVYSMALVLWEMTSRCNA |
| | VGEVKDYEPPFGSKVREHPCVESMKDNVLRDRG |
| | RPEIPSFWLNHQGIQMVCETLTECWDHDPEARL |
| | TAQCVAERFSELEHLDRLSGRSCSEEKIPEDGS |
| | LNTTK |
| | (SEQ ID NO: 4) |
| Human | VTGISLLPPLGVAISVIIIFY |
| TGFβRII | (SEQ ID NO: 5) |
| trans- | |
| membrane | |
| domain | |
| Human | TIPPHVQKSVNNDMIVTDNNGAVKFPQLCKFCD |
| TGFβRII | VRFSTCDNQKSCMSNCSITSICEKPQEVCVAVW |
| extra- | RKNDENITLETVCHDPKLPYHDFILEDAASPKC |
| cellular | IMKEKKKPGETFFMCSCSSDECNDNIIFSEEYN |
| domain | TSNPDLLLVIFQ |
| | (SEQ ID NO: 6) |

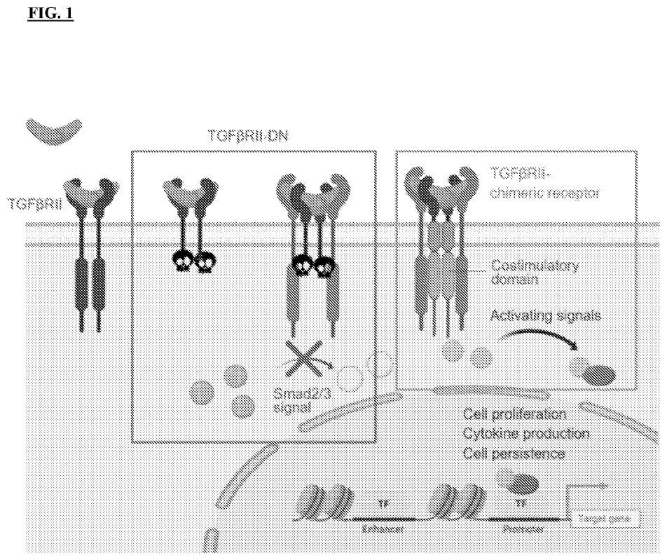

Binding of TGFβ family ligands to TGFβRII results in the recruitment of TGFβRI and formation of a stable oligomeric receptor complex, composed of two TGFβRI and two TGFβRII molecules symmetrically bound to the cytokine dimer (see FIG. 1). TGFβRI is activated by phosphorylation by the constitutively active TGFβRII. The binding of active TGFβ to the receptor complex triggers receptor serine/threonine kinase activity, allowing for the phosphorylation of downstream signaling targets. TGFβ signaling through its cognate receptor initiates canonical and non-canonical signaling pathways. In the canonical pathway, activated TGFβRI phosphorylates SMAD2/3, which dissociates from the receptor and interacts with SMAD4. The SMAD2/3-SMAD4 complex is subsequently translocated to the nucleus where it modulates the transcription of the TGFβ regulated genes. Additionally, TGFβ activates different non-SMAD pathways, e.g., the non-canonical signaling pathway, including PI3K, Ras, Par6, and Jnk/p38/MAPK pathways.

Many cancers, particularly prostate cancer, are known to secrete TGFβ, creating an immunosuppressive milieu. TGFβ is known to induce or promote metastasis and neoangiogenesis and to potently suppress the immune system. Furthermore, TGFβ inhibits proliferation and cytokine secretion on resting CD4 T cells.

Various means of inhibiting TGFβ signaling have been described, including using a dominant-negative TGFβRII (dnTGFβRII), which is truncated and lacks the intracellular kinase domain necessary for downstream signaling. See, e.g., Kloss et al., Molecular Therapy 26(7):1855-66 (2018).

As used herein, the term “immune cell” refers to a cell of the immune system. In some aspects, the immune cell is selected from a T lymphocyte (“T cell”), B lymphocyte (“B cell”), natural killer (NK) cell, natural killer T (NKT) cell, macrophage, eosinophil, mast cell, dendritic cell or neutrophil). As used herein, the terms “T cell” and “T lymphocyte” are interchangeable and refer to any lymphocytes produced or processed by the thymus gland. Non-limiting classes of T cells include effector T cells and Th cells (such as CD4⁺ or CD8⁺ T cells). In some aspects, the immune cell is a Th1 cell. In some aspects, the immune cell is a Th2 cell. In some aspects, the immune cell is a Tc17 cell. In some aspects, the immune cell is a Th17 cell. In some aspects, the immune cell is a tumor-infiltrating cell (TIL). In some aspects, the immune cell is a Tre g cell. As used herein, an “immune cell” also refers to a pluripotent cell, e.g., a stem cell (e.g., an embryonic stem cell or a hematopoeitc stem cell) or an induced pluripotent stem cell, which is capable of differentiation into an immune cell.

In some aspects, the T cell is a memory T cell. As used herein, the term “memory” T cells refers to T cells that have previously encountered and responded to their cognate antigen (e.g., in vivo, in vitro, or ex vivo) or which have been stimulated with, e.g., an anti-CD3 antibody (e.g., in vitro or ex vivo). Immune cells having a “memory-like” phenotype upon secondary exposure, such memory T cells can reproduce to mount a faster and strong immune response than during the primary exposure. In some aspects, memory T cells comprise central memory T cells (T_{CM} cells), effector memory T cells (TEM cells), tissue resident memory T cells (Tim cells), stem cell-like memory T cells (T_{SCM} cells), or any combination thereof.

In some aspects, the T cell is a stem cell-like memory T cell. As used herein, the term “stem cell-like memory T cells,” “T memory stem cells,” or “T_{SCM} cells” refer to memory T cells that express CD95, CD45RA, CCR7, and CD62L and are endowed with the stem cell-like ability to self-renew and the multipotent capacity to reconstitute the entire spectrum of memory and effector subsets.

In some aspects, the T cell is a central memory T cell. As used herein, the term “central memory T cells” or “T_{CM} cells” refer to memory T cells that express CD45RO, CCR7, and CD62L. Central memory T cells are generally found within the lymph nodes and in peripheral circulation.

In some aspects, the T cell is an effector memory T cell. As used herein, the term “effector memory T cells” or “TEM cells” refer to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

In some aspects, the T cell is a tissue resident memory T cell. As used herein, the term “tissue resident memory T cells” or “MINA cells” refer to memory T cells that do not circulate and remain resident in peripheral tissues, such as the skin, lung, and the gastrointestinal tract. In certain aspects, tissue resident memory T cells are also effector memory T cells.

In some aspects, the T cell is a naïve T cell. As used herein, the term “naïve T cells” or “T_{N} cells” refers to T cells that express CD45RA, CCR7, and CD62L, but which do not express CD95. T_{N} cells represent the most undifferentiated cell in the T cell lineage. The interaction between a T_{N} cell and an antigen presenting cell (APC) induces differentiation of the T_{N} cell towards an activated T_{EFF} cell and an immune response. In some aspects, the T cell is an effector T (T_{eff}) cell.

As used herein, “cell engineering” refers to the targeted modification of a cell, e.g., an immune cell disclosed herein. In some aspects, the cell engineering comprises viral genetic engineering, non-viral genetic engineering, introduction of receptors to allow for tumor specific targeting (e.g., a chimeric antigen receptor (CAR) or a T cell receptor (TCR)) introduction of one or more endogenous genes that improve T cell function, introduction of one or more synthetic genes that improve immune cell, e.g., T cell, function (e.g., a chimeric activation receptor disclosed herein), or any combination thereof.

As used herein, the term “cytokine” refers to small, secreted proteins released by cells that have a specific effect on the interactions and communications between cells. Non-limiting examples of cytokines include interleukins (e.g., interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21 and IL-23), interferons (IFN; e.g., IFNα, IFNβ, and IFNγ), tumor necrosis factor (TNF) family members, and transforming growth factor (TGF) family members. In certain aspects of the present disclosure, the interaction between TGFβ and a chimeric activation receptor disclosed herein in a host immune cell leads to increased expression of one or more cytokine. In some aspects, the cytokine is an interleukin. In some aspects, the cytokine is selected from IL-2, IL-7, IL-15, IL-21 and any combination thereof. In particular aspects, the cytokine is IL-2. IL-2 (UniProtKB—P60568) is produced by T cells in response to antigenic or mitogenic stimulation. IL-2 is known to stimulate T cell proliferation and other activities crucial to regulation of the immune response.

In some aspects, the cytokine is an interferon. In some aspects, the interferon is selected from IFNα, IFNβ, and IFNγ. In certain aspects, the interferon is IFNγ. IFNγ (UniProtKB—P01579) is produced by activated lymphocytes promoted immune cell function.

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein (e.g., an immune cell described herein) include intravenous, intraperitoneal, intramuscular, subcutaneous, spinal or other parenteral routes of administration, for example by injection or infusion.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricular, intravitreal, epidural, and intrasternal injection and infusion, as well as in vivo electroporation.

Alternatively, a therapeutic agent described herein (e.g., an immune cell described herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell).

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, NKT cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the terms “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

As used herein, the term “anti-tumor immune response” refers to an immune response against a tumor antigen.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein. As used herein, the phrase “subject in need thereof” includes subjects, such as mammalian subjects, that would benefit, e.g., from administration of immune cells, e.g., T cells, as described herein to control tumor growth.

The term “effective amount” or “effective dosage” refers to an amount of an agent (e.g., an immune cell disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., immune cells as described herein) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

The terms “effective” and “effectiveness” with regard to a treatment include both pharmacological effectiveness and physiological safety. Pharmacological effectiveness refers to the ability of a composition disclosed herein (e.g., immune cells described herein) to promote cancer regression in the patient. Physiological safety refers to the level of toxicity, or other adverse physiological effects at the cellular, organ, and/or organism level (adverse effects) resulting from administration of a composition disclosed herein (e.g., immune cells as described herein).

The term “chimeric activation receptor,” as used herein, refers to a recombinant fusion protein comprising an extracellular ligand-binding receptor, a transmembrane domain, and an intracellular signaling domain (e.g., an intracellular costimulatory domain), wherein the extracellular ligand-binding receptor and the intracellular signaling domain are not derived from the same protein. In some aspects, the extracellular ligand-binding receptor comprises the extracellular domain of a TGFβ receptor or a fragment thereof, wherein the fragment of the TGFβ receptor retains the ability to interact with TGFβ. In some aspects, the extracellular ligand-binding receptor comprises an antigen-binding domain that specifically binds TGFβ.

The terms “chimeric antigen receptor” and “CAR,” as used herein, refer to a recombinant fusion protein that has an antigen-specific extracellular domain coupled to an intracellular domain that directs the cell to perform a specialized function upon binding of an antigen to the extracellular domain. In some aspects, a chimeric antigen receptor disclosed herein comprises a chimeric polypeptide of the present disclosure.

The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen-binding agents by their ability to both bind MHC-independent antigen and transduce activation signals via their intracellular domain.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (Ku) between about 0.1 pM to about 10 μM, for example, about 0.1 pM to about 1 μM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains such as cAb VHH (camelid antibody variable domains) and humanized versions thereof, lgNAR VH (shark antibody variable domains) and humanized versions thereof, sdAb VH (single domain antibody variable domains), and “camelized” antibody variable domains are also suitable for use in a CAR of the present disclosure. In some aspects, T cell receptor (TCR) based recognition domains, such as single chain TCR (scTv, i.e., single chain two-domain TCR containing VαVβ) are also suitable for use in a TCR of the present disclosure.

As used herein, a “transforming growth factor β (TGFβ)-binding domain” refers to a polypeptide which is capable of binding TGFβ. In some aspects, the TGFβ-binding domain comprises a binding domain selected from a single chain Fv (scFv), a cAb VHH and humanized versions thereof, lgNAR VH and humanized versions thereof, an sdAb VH, “camelized” antibody variable domains, a T cell receptor (TCR) based recognition domains (such as single chain TCR (scTv, i.e., single chain two-domain TCR containing VαVβ)), and any combinations thereof. In some aspects, the TGFβ-binding domain comprises an extracellular domain of a TGFβ receptor, e.g., a human TGFβ receptor. In some aspects, the TGFβ-binding domain comprises an extracellular domain of human TGFβRI. In some aspects, the TGFβ-binding domain comprises a fragment of the extracellular domain of human TGFβRI, which retains the ability to interact with TGFβ. In some aspects, the TGFβ-binding domain comprises an extracellular domain of human TGFβRII. In some aspects, the TGFβ-binding domain comprises a fragment of the extracellular domain of human TGFβRII, which retains the ability to interact with TGFβ.

As used herein, a “costimulatory domain” refers to a polypeptide that stimulates an immune response. In some aspects, the costimulatory domain comprises or is derived from a naturally occurring costimulatory receptor. During an immune response, costimulatory signals by costimulatory receptors enhance T cell proliferation, cytokine secretion, cytotoxic function, memory formation or survival. In certain aspects, the costimulatory domain comprises an intracellular fragment of a costimulatory receptor, wherein the fragment retains the ability to propagate a costimulatory signal. In some aspects, the costimulatory receptor is selected from CD2, CD8, CD28, 4-1BB, ICOS, OX40, DAP10, TNFR1A, TNFR1B, DR3, TNFR2, CD27, HVEM, GITR, CD40, and any combination thereof. In certain aspects, the costimulatory domain comprises an intracellular fragment of CD2, wherein the intracellular fragment of CD2 is capable of propagating a costimulatory signal. In particular aspects, the costimulatory domain comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 (Table 2). In certain aspects, the costimulatory domain comprises the amino acid sequence set forth in SEQ ID NO: 7 or a functional fragment thereof. In certain aspects, the costimulatory domain comprises an intracellular fragment of CD28, wherein the intracellular fragment of CD28 is capable of propagating a costimulatory signal. In particular aspects, the costimulatory domain comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16 (Table 2). In certain aspects, the costimulatory domain comprises the amino acid sequence set forth in SEQ ID NO: 16 or a functional fragment thereof. In certain aspects, the costimulatory domain comprises an intracellular fragment of OX40, wherein the intracellular fragment of OX40 is capable of propagating a costimulatory signal. In particular aspects, the costimulatory domain comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17 (Table 2). In certain aspects, the costimulatory domain comprises the amino acid sequence set forth in SEQ ID NO: 17 or a functional fragment thereof.

TABLE 2
Costimulatory Domain Amino Acid Sequences

| | |
|---|---|
| CD2 | KRKKQRSRRNDEELETRAHRVATEERGRKPHQIPA |
| Costimu- | STPQNPATSQHPPPPPGHRSQAPSHRPPPPGHRVQ |
| latory | HQPQKRPPAPSGTQVHQQKGPPLPRPRVQPKPPHG |
| Domain | AAENSLSPSSN |
| | (SEQ ID NO: 7) |
| CD28 | RSKRSRLLHSDYMNMTPRRPGPTRKHYQPYAPPRD |
| costimu- | FAAYRS |
| latory | (SEQ ID NO: 16) |
| domain | |
| OX40 | VAAILGLGLVLGLLGPLAILLALYLLRRDQRLPPD |
| costimu- | AHKPPGGGSFRTPIQEEQADAHSTLAKI |
| latory | (SEQ ID NO: 17) |
| domain | |

As used herein, the term “T cell receptor” or “TCR” refers to a heterodimer composed of 2 different transmembrane polypeptide chains: an α chain and a β chain, each consisting of a constant region, which anchors the chain inside the T-cell surface membrane, and a variable region, which recognizes and binds to the antigen presented by MHCs. The TCR complex is associated with 6 polypeptides forming 2 heterodimers, CD3γε and CD3δε, and 1 homodimer CD3 ζ, which together forms the CD3 complex. T-cell receptor-engineered T-cell therapy utilizes the modification of T cells that retain these complexes to specifically target the antigens expressed by particular tumor cells. As used herein, the term “TCR” includes naturally occurring TCRs and engineered TCRs.

As used herein, an “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of immune cells, e.g., T cells, NK cells, and/or TILs.

A “TCR mimic” or a “TCRm” refers to a type of antibody that recognize epitopes comprising both the peptide and the MHC-I molecule, similar to the recognition of such complexes by the TCR on T cells.

As used herein, the terms “ug” and “uM” are used interchangeably with “μg” and “μM” respectively.

Various aspects described herein are described in further detail in the following subsections.

### II. Compositions of the Disclosure

Certain aspects of the present disclosure are directed to chimeric activation receptors comprising (i) a transforming growth factor β (TGFβ)-binding domain; (ii) a transmembrane domain; (iii) and a CD2 costimulatory domain. In some aspects, the chimeric activation receptor is capable of competing with an endogenous TGFβRI and/or an endogenous TGFβRII for binding to TGFβ. In some aspects, the TGFβ-binding domain comprises an extracellular domain of TGFβRI or a fragment thereof, wherein the fragment retains the ability to interact with TGFβ. In some aspects, the TGFβRI is a human TGFβRI. In some aspects, the TGFβ-binding domain comprises an extracellular domain of TGFβRII or a fragment thereof, wherein the fragment retains the ability to interact with TGFβ. In some aspects, the TGFβRII is a human TGFβRI.

Other aspects of the present disclosure are directed to a nucleic acid molecule encoding a chimeric activation receptor disclosed herein. In some aspects, the nucleic acid molecule further encodes a chimeric antigen receptor (CAR) and/or a T cell receptor (TCR). Other aspects of the present disclosure are directed to an immune cell comprising a chimeric activation receptor disclosed herein or a nucleic acid molecule disclosed herein. In some aspects, the immune cell further comprises a CAR and/or a TCR.

II.A. Polypeptides

Certain aspects of the present disclosure are directed to a chimeric activation receptor comprising (i) a TGFβ-binding domain; (ii) a transmembrane domain; (iii) and a CD2 costimulatory domain. In some aspects, the chimeric activation receptor is capable of competing with an endogenous TGFβR for binding to TGFβ. In some aspects, the chimeric activation receptor is capable of competing with an endogenous TGFβRI for binding to TGFβ. In some aspects, the chimeric activation receptor is capable of competing with an endogenous TGFβRI for binding to TGFβ.

In certain aspects, the chimeric activation receptor is capable of interacting with both TGFβ and an endogenous TGFβ receptor. In some aspects, the chimeric activation receptor has a higher affinity for TGFβ, e.g., human TGFβ, than an endogenous TGFβR. In some aspects, the chimeric activation receptor has an affinity at least about 2-fold, at least about 2.5-fold, at least about 3-fold, at least about 3.5-fold, at least about 4-fold, at least about 4-fold, at least about 4.5-fold, at least about 5-fold, at least about 6-fold, at least about 7-fold, at least about 8-fold, at least about 9-fold, at least about 10-fold greater than the affinity of an endogenous TGFβR. In some aspects, the chimeric activation receptor comprises an extracellular domain of a human TGFβRI, or a TGFβ-binding portion thereof, and the chimeric activation receptor has an affinity at least about 2-fold, at least about 2.5-fold, at least about 3-fold, at least about 3.5-fold, at least about 4-fold, at least about 4-fold, at least about 4.5-fold, at least about 5-fold, at least about 6-fold, at least about 7-fold, at least about 8-fold, at least about 9-fold, at least about 10-fold greater than the affinity of an endogenous TGFβRI for TGFβ. In some aspects, the chimeric activation receptor comprises an extracellular domain of a human TGFβRII, or a TGFβ-binding portion thereof, and the chimeric activation receptor has an affinity at least about 2-fold, at least about 2.5-fold, at least about 3-fold, at least about 3.5-fold, at least about 4-fold, at least about 4-fold, at least about 4.5-fold, at least about 5-fold, at least about 6-fold, at least about 7-fold, at least about 8-fold, at least about 9-fold, at least about 10-fold greater than the affinity of an endogenous TGFβRII for TGFβ.

In some aspects, the chimeric activation receptor is capable of interacting with TGFβ and an endogenous TGFβRI. In certain aspects, the chimeric activation receptor comprises an extracellular domain of a human TGFβRII, or a TGFβ-binding portion thereof, and the chimeric activation receptor is capable of interacting with TGFβ and an endogenous TGFβRI. In some aspects, the chimeric activation receptor is capable of forming a heterotetradimer with an endogenous TGFβRI. In some aspects, upon binding to TGFβ, the chimeric activation receptor has a higher affinity for an endogenous TGFβRI than a naturally occurring TGFβRII has for TGFβRI. In some aspects, the chimeric activation receptor has an affinity at least about 2-fold, at least about 2.5-fold, at least about 3-fold, at least about 3.5-fold, at least about 4-fold, at least about 4-fold, at least about 4.5-fold, at least about 5-fold, at least about 6-fold, at least about 7-fold, at least about 8-fold, at least about 9-fold, at least about 10-fold greater than the affinity of an endogenous TGFβRII has for endogenous TGFβRI.

In some aspects, the chimeric activation receptor is capable of interacting with TGFβ and an endogenous TGFβRII. In certain aspects, the chimeric activation receptor comprises an extracellular domain of a human TGFβRI, or a TGFβ-binding portion thereof, and the chimeric activation receptor is capable of interacting with TGFβ and an endogenous TGFβRII. In some aspects, the chimeric activation receptor is capable of forming a heterotetradimer with an endogenous TGFβRII. In some aspects, upon binding to TGFβ, the chimeric activation receptor has a higher affinity for an endogenous TGFβRII than a naturally occurring TGFβRI has for TGFβRII. In some aspects, the chimeric activation receptor has an affinity at least about 2-fold, at least about 2.5-fold, at least about 3-fold, at least about 3.5-fold, at least about 4-fold, at least about 4-fold, at least about 4.5-fold, at least about 5-fold, at least about 6-fold, at least about 7-fold, at least about 8-fold, at least about 9-fold, at least about 10-fold greater than the affinity of an endogenous TGFβRI has for endogenous TGFβRII.

Without being bound by any particular mechanism, the chimeric activation receptors disclosed herein are able to convert an inhibitory TGFβ signal into an activation signal in immune cells in the tumor microenvironment. The enhancement of the immune response in a cell (e.g., an immune cell) expressing the chimeric activation receptor can occur by a number of different mechanisms. In some aspects, the interaction of TGFβ with the chimeric activation receptor induces the expression (i.e., production) of one or more pro-immune factors in a cell expressing the chimeric activation receptor (e.g., an immune cell). In some aspects, upon interaction of the chimeric activation receptor with TGFβ, the cell (e.g., the immune cell) produces one or more cytokines. In some aspects, upon interaction of the chimeric activation receptor with TGFβ, the cell (e.g., the immune cell) upregulates the expression and/or production of one or more cytokines. In some aspects, the cytokine is produced (e.g., expressed) at a higher level than a similar cell (e.g., immune cell) that does not comprise the chimeric activation receptor upon interaction with TGFβ. In certain aspects, the cytokine is produced (e.g., expressed) at a higher level than a similar cell (e.g., immune cell) expressing a TGFβRII-binding domain fused to a CD28 costimulatory domain upon interaction with TGFβ.

In certain aspects, the cytokine is an interleukin. In some aspects, the interleukin is selected from ILL IL2, IL4, IL5, IL6, IL7, IL-9, IL-10, IL12, IL13, IL15, IL17, IL18, IL21, IL23, IL36, EPO, TPO and any combination thereof. In some aspects, the one or more cytokines comprise IL2. In certain aspects, the cytokine is an interferon. In some aspects, the one or more cytokines comprise IFNγ. In certain aspects, the cytokine is an interferon. In some aspects, the one or more cytokines comprise IL2 and IFNγ. In certain aspects, the cytokine comprises TNFa. In certain aspects, the cytokine comprises GMCSF.

In some aspects, upon interaction with TGFβ, a cell (e.g., an immune cell) that expresses the chimeric activation receptor expresses IL2 at a level that is at least about 125%, at least about 150%, at least about 200%, at least about 250%, at least about 300%, at least about 350%, at least about 400%, at least about 450%, at least about 500% the expression of IL2 by a similar cell (e.g., immune cell) that does not comprise the chimeric activation receptor upon interaction with TGFβ. In some aspects, the similar cell (e.g., immune cell) that does not comprise the chimeric activation receptor comprises an activation receptor having a CD28 costimulatory domain. In some aspects, upon interaction with TGFβ, a cell (e.g., an immune cell) that expresses the chimeric activation receptor expresses IL2 at a level that is at least about 125%, at least about 150%, at least about 200%, at least about 250%, at least about 300%, at least about 350%, at least about 400%, at least about 450%, at least about 500% the expression of IL2 by a similar cell (e.g., immune cell) comprising a dominant negative TGFβRI and/or dominant negative TGFβRII upon interaction with TGFβ. In some aspects, upon interaction with TGFβ, a cell (e.g., an immune cell) that expresses the chimeric activation receptor expresses IL2 at a level that is at least about 125%, at least about 150%, at least about 200%, at least about 250%, at least about 300%, at least about 350%, at least about 400%, at least about 450%, at least about 500% the expression of IL2 by a similar cell (e.g., immune cell) expressing a TGFβRII-binding domain with no intracellular domain upon binding to TGFβ. In some aspects, upon interaction with TGFβ, a cell (e.g., an immune cell) that expresses the chimeric activation receptor expresses IL2 at a level that is at least about 125%, at least about 150%, at least about 200%, at least about 250%, at least about 300%, at least about 350%, at least about 400%, at least about 450%, at least about 500% the expression of IL2 by a similar cell (e.g., immune cell) expressing a TGFβRII-binding domain fused to a CD28 costimulatory domain upon binding to TGFβ. In certain aspects, the IL2 expression is at least about 150%. In certain aspects, the IL2 expression is at least about 200%. In certain aspects, the IL2 expression is at least about 250%. In certain aspects, the IL2 expression is at least about 300%. In certain aspects, the IL2 expression is at least about 350%. In certain aspects, the IL2 expression is at least about 400%. In certain aspects, the IL2 expression is at least about 450%. In certain aspects, the IL2 expression is at least about 500%. In certain aspects, the IL2 expression is at least about 750%. In certain aspects, the IL2 expression is at least about 1000%. In some aspects, the similar cell (e.g., immune cell) that does not comprise the chimeric activation receptor comprises an activation receptor having a CD28 costimulatory domain.

In some aspects, upon interaction with TGFβ, a cell (e.g., an immune cell) that expresses the chimeric activation receptor expresses IL2 at a level that is between about 1.5-fold and about 20-fold higher than the expression of IL2 by a similar cell (e.g., immune cell) that does not comprise the chimeric activation receptor upon interaction with TGFβ. In some aspects, upon interaction with TGFβ, a cell (e.g., an immune cell) that expresses the chimeric activation receptor expresses IL2 at a level that is at least about 1.25-fold, at least about 1.5-fold, at least about 2-fold, at least about 2.5-fold, at least about 3-fold, at least about 3.5-fold, at least about 4-fold, at least about 4.5-fold, at least about 5-fold, at least about 10-fold, at least about 15-fold, or at least about higher than the expression of IL2 by a similar cell (e.g., immune cell) that does not comprise the chimeric activation receptor upon interaction with TGFβ. In some aspects, the similar cell (e.g., immune cell) that does not comprise the chimeric activation receptor comprises an activation receptor having a CD28 costimulatory domain. In some aspects, upon interaction with TGFβ, a cell (e.g., an immune cell) that expresses the chimeric activation receptor expresses IL2 at a level that is at least about 1.25-fold, at least about 1.5-fold, at least about 2-fold, at least about 2.5-fold, at least about 3-fold, at least about 3.5-fold, at least about 4-fold, at least about 4.5-fold, or at least about 5-fold higher than the expression of IL2 by a similar cell (e.g., immune cell) comprising a dominant negative TGFβRI and/or dominant negative TGFβRII upon interaction with TGFβ. In some aspects, upon interaction with TGFβ, a cell (e.g., an immune cell) that expresses the chimeric activation receptor expresses IL2 at a level that is at least about 1.25-fold, at least about 1.5-fold, at least about 2-fold, at least about 2.5-fold, at least about 3-fold, at least about 3.5-fold, at least about 4-fold, at least about 4.5-fold, or at least about 5-fold higher than the expression of IL2 by a similar cell (e.g., immune cell) expressing a TGFβRII-binding domain with no intracellular domain upon binding to TGFβ. In some aspects, upon interaction with TGFβ, a cell (e.g., an immune cell) that expresses the chimeric activation receptor expresses IL2 at a level that is at least about 1.25-fold, at least about 1.5-fold, at least about 2-fold, at least about 2.5-fold, at least about 3-fold, at least about 3.5-fold, at least about 4-fold, at least about 4.5-fold, at least about higher, at least about 10-fold higher, at least about 15-fold, or at least about 20-fold higher than the expression of IL2 by a similar cell (e.g., immune cell) expressing a TGFβRII-binding domain fused to a CD28 costimulatory domain upon binding to TGFβ. In certain aspects, the IL2 expression is increased by at least about 1.5-fold. In certain aspects, the IL2 expression is increased by at least about 2-fold. In certain aspects, the IL2 expression is increased by at least about 2.5-fold. In certain aspects, the IL2 expression is increased by at least about 3-fold. In certain aspects, the IL2 expression is increased by at least about 3.5-fold. In certain aspects, the IL2 expression is increased by at least about 4-fold. In certain aspects, the IL2 expression is increased by at least about 4.5-fold. In certain aspects, the IL2 expression is increased by at least about 5-fold. In certain aspects, the IL2 expression is increased by at least about 7.5-fold. In certain aspects, the IL2 expression is increased by at least about 10-fold. In certain aspects, the IL2 expression is increased by at least about 15-fold. In certain aspects, the IL2 expression is increased by at least about 20-fold.

In some aspects, upon interaction with TGFβ, a cell (e.g., an immune cell) that expresses the chimeric activation receptor expresses IFNγ at a level that is at least about 125%, at least about 150%, at least about 200%, at least about 250%, at least about 300%, at least about 350%, at least about 400%, at least about 450%, at least about 500% the expression of IFNγ by a similar cell (e.g., immune cell) that does not comprise the chimeric activation receptor upon interaction with TGFβ. In some aspects, the similar cell (e.g., immune cell) that does not comprise the chimeric activation receptor comprises an activation receptor having a CD28 costimulatory domain. In some aspects, upon interaction with TGFβ, a cell (e.g., an immune cell) that expresses the chimeric activation receptor expresses IFNγ at a level that is at least about 125%, at least about 150%, at least about 200%, at least about 250%, at least about 300%, at least about 350%, at least about 400%, at least about 450%, at least about 500% the expression of IFNγ by a similar cell (e.g., immune cell) comprising a dominant negative TGFβRI and/or dominant negative TGFβRII upon interaction with TGFβ. In some aspects, upon interaction with TGFβ, a cell (e.g., an immune cell) that expresses the chimeric activation receptor expresses IFNγ at a level that is at least about 125%, at least about 150%, at least about 200%, at least about 250%, at least about 300%, at least about 350%, at least about 400%, at least about 450%, at least about 500% the expression of IFNγ by a similar cell (e.g., immune cell) expressing a TGFβRII-binding domain with no intracellular domain upon binding to TGFβ. In some aspects, upon interaction with TGFβ, a cell (e.g., an immune cell) that expresses the chimeric activation receptor expresses IFNγ at a level that is at least about 125%, at least about 150%, at least about 200%, at least about 250%, at least about 300%, at least about 350%, at least about 400%, at least about 450%, at least about 500% the expression of IFNγ by a similar cell (e.g., immune cell) expressing a TGFβRII-binding domain fused to a CD28 costimulatory domain upon binding to TGFβ. In certain aspects, the IFNγ expression is at least about 150%. In certain aspects, the IFNγ expression is at least about 200%. In certain aspects, the IFNγ expression is at least about 250%. In certain aspects, the IFNγ expression is at least about 300%. In certain aspects, the IFNγ expression is at least about 350%. In certain aspects, the IFNγ expression is at least about 400%. In certain aspects, the IFNγ expression is at least about 450%. In certain aspects, the IFNγ expression is at least about 500%. In certain aspects, the IFNγ expression is at least about 750%. In certain aspects, the IFNγ expression is at least about 1000%.

In some aspects, upon interaction with TGFβ, a cell (e.g., an immune cell) that expresses the chimeric activation receptor expresses IFNγ at a level that is between about 1.5-fold and about 20-fold higher than the expression of IFNγ by a similar cell (e.g., immune cell) that does not comprise the chimeric activation receptor upon interaction with TGFβ. In some aspects, upon interaction with TGFβ, a cell (e.g., an immune cell) that expresses the chimeric activation receptor expresses IFNγ at a level that is at least about 1.25-fold, at least about 1.5-fold, at least about 2-fold, at least about 2.5-fold, at least about 3-fold, at least about 3.5-fold, at least about 4-fold, at least about 4.5-fold, at least about 5-fold, at least about 8-fold, at least about 10-fold, at least about 12-fold, at least about 15-fold, at least about 18-fold, or at least about 20-fold higher than the expression of IFNγ by a similar cell (e.g., immune cell) that does not comprise the chimeric activation receptor upon interaction with TGFβ. In some aspects, the similar cell (e.g., immune cell) that does not comprise the chimeric activation receptor comprises an activation receptor having a CD28 costimulatory domain. In some aspects, upon interaction with TGFβ, a cell (e.g., an immune cell) that expresses the chimeric activation receptor expresses IFNγ at a level that is at least about 1.25-fold, at least about 1.5-fold, at least about 2-fold, at least about 2.5-fold, at least about 3-fold, at least about 3.5-fold, at least about 4-fold, at least about 4.5-fold, or at least about 5-fold higher than the expression of IFNγ by a similar cell (e.g., immune cell) comprising a dominant negative TGFβRI and/or dominant negative TGFβRII upon interaction with TGFβ. In some aspects, upon interaction with TGFβ, a cell (e.g., an immune cell) that expresses the chimeric activation receptor expresses IFNγ at a level that is at least about 1.25-fold, at least about 1.5-fold, at least about 2-fold, at least about 2.5-fold, at least about 3-fold, at least about 3.5-fold, at least about 4-fold, at least about 4.5-fold, at least about 5-fold higher than the expression of IFNγ by a similar cell (e.g., immune cell) expressing a TGFβRII-binding domain with no intracellular domain upon binding to TGFβ. In some aspects, upon interaction with TGFβ, a cell (e.g., an immune cell) that expresses the chimeric activation receptor expresses IFNγ at a level that is at least about 1.25-fold, at least about 1.5-fold, at least about 2-fold, at least about 2.5-fold, at least about 3-fold, at least about 3.5-fold, at least about 4-fold, at least about 4.5-fold, at least about 5-fold higher than the expression of IFNγ by a similar cell (e.g., immune cell) expressing a TGFβRII-binding domain fused to a CD28 costimulatory domain upon binding to TGFβ. In certain aspects, the IFNγ expression is increased by at least about 1.5-fold. In certain aspects, the IFNγ expression is increased by at least about 2-fold. In certain aspects, the IFNγ expression is increased by at least about 2.5-fold. In certain aspects, the IFNγ expression is increased by at least about 3-fold. In certain aspects, the IFNγ expression is increased by at least about 3.5-fold. In certain aspects, the IFNγ expression is increased by at least about 4-fold. In certain aspects, the IFNγ expression is increased by at least about 4.5-fold. In certain aspects, the IFNγ expression is increased by at least about 5-fold. In certain aspects, the IFNγ expression is increased by at least about 7.5-fold. In certain aspects, the IFNγ expression is increased by at least about 10-fold. In certain aspects, the IFNγ expression is increased by at least about 12-fold. In certain aspects, the IFNγ expression is increased by at least about 14-fold. In certain aspects, the IFNγ expression is increased by at least about 15-fold. In certain aspects, the IFNγ expression is increased by at least about 20-fold.

The increased expression of the one or more cytokines can occur any time after the initial interaction between the cell (e.g., immune cell) expressing the chimeric activation receptor and TGFβ, and the upregulation of the one or more cytokines can persist for several hours or several days. In some aspects, the upregulation of the one or more cytokines will persist for as long as TGFβ remains available for the cell. In some aspects, the increased expression of the one or more cytokines by the cell (e.g., immune cell) expressing the chimeric activation receptor (e.g., as compared to a similar cell (i) not comprising the chimeric activation receptor, (ii) expressing a dominant negative TGFβ receptor, (iii) expressing a TGFβRII-binding with no intracellular domain), or (iv) expressing a TGFβRII-binding domain fused to a CD28 costimulatory domain) is observed at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

In some aspects, upon interaction with TGFβ, the cell (e.g., immune cell) expressing the chimeric activation receptor has increased proliferation. In some aspects, upon interaction with TGFβ, the cell (e.g., immune cell) expressing the chimeric activation receptor is more proliferative than a similar cell (e.g., immune cell) that does not comprise the chimeric activation receptor upon interaction with TGFβ. In some aspects, upon interaction with TGFβ, the cell (e.g., immune cell) expressing the chimeric activation receptor is more proliferative than a similar cell (e.g., immune cell) comprising a dominant negative TGFβ receptor upon interaction with TGFβ. In some aspects, upon interaction with TGFβ, the cell (e.g., immune cell) expressing the chimeric activation receptor is more proliferative than a similar cell (e.g., immune cell) comprising a TGFβRII-binding with no intracellular domain upon interaction with TGFβ In some aspects, upon interaction with TGFβ, the cell (e.g., immune cell) expressing the chimeric activation receptor is more proliferative than a similar cell (e.g., immune cell) comprising a TGFβRII-binding domain fused to a CD28 costimulatory domain upon interaction with TGFβ. In some aspects, cell proliferation is increased by at least about 25%, at least about 50%, at least about 75%, at least about 100%, at least about 125%, at least about 150%, at least about 175%, at least about 200%, at least about 250%, at least about 300%, at least about 350%, at least about 400%, at least about 450%, at least about 500%, at least about 600%, at least about 700%, at least about 800%, at least about 900%, or at least about 1000%. In some aspects, cell proliferation is increased by at least about 25%. In some aspects, cell proliferation is increased by at least about 50%. In some aspects, cell proliferation is increased by at least about 75%. In some aspects, cell proliferation is increased by at least about 100%. In some aspects, cell proliferation is increased by at least about 125%. In some aspects, cell proliferation is increased by at least about 150%. In some aspects, cell proliferation is increased by at least about 200%. In some aspects, cell proliferation is increased by at least about 250%. In some aspects, cell proliferation is increased by at least about 300%. In some aspects, cell proliferation is increased by at least about 350%. In some aspects, cell proliferation is increased by at least about 400%. In some aspects, cell proliferation is increased by at least about 450%. In some aspects, cell proliferation is increased by at least about 500%. In some aspects, cell proliferation is increased by at least about 600%. In some aspects, cell proliferation is increased by at least about 700%. In some aspects, cell proliferation is increased by at least about 800%. In some aspects, cell proliferation is increased by at least about 900%. In some aspects, cell proliferation is increased by at least about 1000%.

In some aspects, cell proliferation is increased by at least about 1.25-fold, at least about 1.5-fold, at least about 1.75-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about at least about 15-fold, at least about 20-fold, at least about 30-fold, at least about 50-fold, or at least about 100-fold. In some aspects, cell proliferation is increased by at least about 1.25-fold. In some aspects, cell proliferation is increased by at least about 1.5-fold. In some aspects, cell proliferation is increased by at least about 2-fold. In some aspects, cell proliferation is increased by at least about 2.5-fold. In some aspects, cell proliferation is increased by at least about 3-fold. In some aspects, cell proliferation is increased by at least about 3.5-fold. In some aspects, cell proliferation is increased by at least about 4-fold. In some aspects, cell proliferation is increased by at least about 4.5-fold. In some aspects, cell proliferation is increased by at least about 5-fold. In some aspects, cell proliferation is increased by at least about 6-fold. In some aspects, cell proliferation is increased by at least about 7-fold. In some aspects, cell proliferation is increased by at least about 8-fold. In some aspects, cell proliferation is increased by at least about 9-fold. In some aspects, cell proliferation is increased by at least about 10-fold.

In some aspects, upon interaction with TGFβ, the increase in cell proliferation is observed at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

In some aspects, upon interaction with TGFβ, the cell (e.g., immune cell) expressing the chimeric activation receptor has increased cytolytic activity. In some aspects, upon interaction with TGFβ, the cell (e.g., immune cell) expressing the chimeric activation receptor has increased cytolytic activity relative to a similar cell (e.g., immune cell) that does not comprise the chimeric activation receptor upon interaction with TGFβ. In some aspects, upon interaction with TGFβ, the cell (e.g., immune cell) expressing the chimeric activation receptor has increased cytolytic activity relative to a similar cell (e.g., immune cell) comprising a dominant negative TGFβ receptor upon interaction with TGFβ. In some aspects, upon interaction with TGFβ, the cell (e.g., immune cell) expressing the chimeric activation receptor has increased cytolytic activity relative to a similar cell (e.g., immune cell) comprising a TGFβRII-binding with no intracellular domain upon interaction with TGFβ. In some aspects, upon interaction with TGFβ, the cell (e.g., immune cell) expressing the chimeric activation receptor has increased cytolytic activity relative to a similar cell (e.g., immune cell) comprising a TGFβRII-binding domain fused to a CD28 costimulatory domain upon interaction with TGFβ. In some aspects, cytolytic activity is increased by at least about 25%, at least about 50%, at least about 75%, at least about 100%, at least about 125%, at least about 150%, at least about 175%, at least about 200%, at least about 250%, at least about 300%, at least about 350%, at least about 400%, at least about 450%, at least about 500%, at least about 600%, at least about 700%, at least about 800%, at least about 900%, or at least about 1000%. In some aspects, cytolytic activity is increased by at least about 25%. In some aspects, cytolytic activity is increased by at least about 50%. In some aspects, cytolytic activity is increased by at least about 75%. In some aspects, cytolytic activity is increased by at least about 100%. In some aspects, cytolytic activity is increased by at least about 125%. In some aspects, cytolytic activity is increased by at least about 150%. In some aspects, cytolytic activity is increased by at least about 200%. In some aspects, cytolytic activity is increased by at least about 250%. In some aspects, cytolytic activity is increased by at least about 300%. In some aspects, cytolytic activity is increased by at least about 350%. In some aspects, cytolytic activity is increased by at least about 400%. In some aspects, cytolytic activity is increased by at least about 450%. In some aspects, cytolytic activity is increased by at least about 500%. In some aspects, cytolytic activity is increased by at least about 600%. In some aspects, cytolytic activity is increased by at least about 700%. In some aspects, cytolytic activity is increased by at least about 800%. In some aspects, cytolytic activity is increased by at least about 900%. In some aspects, cytolytic activity is increased by at least about 1000%.

In some aspects, cytolytic activity is increased by at least about 1.25-fold, at least about 1.5-fold, at least about 1.75-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold. In some aspects, cytolytic activity is increased by at least about 1.25-fold. In some aspects, cytolytic activity is increased by at least about 1.5-fold. In some aspects, cytolytic activity is increased by at least about 2-fold. In some aspects, cytolytic activity is increased by at least about 2.5-fold. In some aspects, cytolytic activity is increased by at least about 3-fold. In some aspects, cytolytic activity is increased by at least about 3.5-fold. In some aspects, cytolytic activity is increased by at least about 4-fold. In some aspects, cytolytic activity is increased by at least about 4.5-fold. In some aspects, cytolytic activity is increased by at least about 5-fold. In some aspects, cytolytic activity is increased by at least about 6-fold. In some aspects, cytolytic activity is increased by at least about 7-fold. In some aspects, cytolytic activity is increased by at least about 8-fold. In some aspects, cytolytic activity is increased by at least about 9-fold. In some aspects, cytolytic activity is increased by at least about 10-fold.

In some aspects, upon interaction with TGFβ, the increase in cytolytic activity is observed at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

II.A.1. Transforming Growth Factor β (TGFβ)-Binding Domain

The chimeric activation receptors disclosed herein comprise at least (i) a TGFβ-binding domain and (ii) a costimulatory domain, e.g., a CD2 costimulatory domain. Any moiety that is capable of binding TGFβ can be used as the TGFβ-binding domain. In some aspects, the TGFβ-binding domain is a polypeptide. TGFβ-binding domain comprises an antibody or an antigen-binding fragment thereof that specifically binds TGFβ. In some aspects, the TGFβ-binding domain comprises a single chain Fv (scFv), which specifically binds TGFβ, e.g., human TGFβ. In some aspects, the TGFβ-binding domain comprises a cAb VHH, which specifically binds TGFβ, e.g., human TGFβ. In some aspects, the cAB VHH is humanized. In some aspects, the TGFβ-binding domain comprises an IgNAR VH (i.e., a VNAR), which specifically binds TGFβ, e.g., human TGFβ. In some aspects, the VNAR is humanized. In some aspects, the TGFβ-binding domain comprises an sdAb VH, which specifically binds TGFβ, e.g., human TGFβ. In some aspects, the TGFβ-binding domain comprises a “camelized” antibody variable domain, which specifically binds TGFβ, e.g., human TGFβ. In some aspects, the TGFβ-binding domain comprises a TCR-based recognition domain (such as single chain TCR (scTv, i.e., single chain two-domain TCR containing VαVβ)), which specifically binds TGFβ, e.g., human TGFβ.

In some aspects, the TGFβ-binding domain comprises an extracellular domain of a TGFβ receptor. In some aspects, the TGFβ-binding domain comprises an extracellular domain of a human TGF receptor.

In some aspects, the TGFβ-binding domain comprises an extracellular domain of human TGFβRI. In some aspects, the TGFβ-binding domain comprises a fragment of the extracellular domain of human TGFβRI, which retains the ability to interact with TGFβ. In some aspects, the TGFβ-binding domain comprises an extracellular domain of human TGFβRII. In some aspects, the TGFβ-binding domain comprises a fragment of the extracellular domain of human TGFβRII, which retains the ability to interact with TGFβ.

In some aspects, the TGFβ-binding domain comprises the extracellular domain of TGFβRI. In some aspects, the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 3. In some aspects, the TGFβ-binding domain comprises the amino acid sequence set forth in SEQ ID NO: 3. In some aspects, the TGFβ-binding domain comprises a fragment of TGFβRI, wherein the fragment is capable of binding TGFβ. In some aspects, the TGFβ-binding domain comprises a fragment of TGFβRI comprising amino acids 34-126 of SEQ ID NO: 1. In some aspects, the TGFβ-binding domain comprises a fragment of TGFβRI comprising amino acids 34-125, amino acids 34-120, amino acids 34-115, amino acids 34-110, amino acids 34-105, amino acids 34-100, amino acids 34-95, amino acids 34-90, amino acids 34-85, amino acids 34-80, amino acids 34-75, amino acids 34-70, amino acids 34-65, amino acids 34-60, amino acids 34-55, or amino acids 34-50 of SEQ ID NO: 1. In some aspects, the TGFβ-binding domain comprises a fragment of TGFβRI comprising amino acids 34-126, amino acids 35-126, amino acids 40-126, amino acids 45-126, amino acids 50-126, amino acids 55-126, amino acids 60-126, amino acids 65-126, amino acids 70-126, amino acids 75-126, amino acids 80-126, amino acids 85-126, amino acids 90-126, amino acids 95-126, or amino acids 100-126 of SEQ ID NO: 1. In some aspects, the TGFβ-binding domain comprises a fragment of TGFβRI comprising amino acids 34-126 of SEQ ID NO: 1. In certain aspects, the TGFβ-binding domain comprises the amino acid sequence set forth in SEQ ID NO: 18. In certain aspects, the TGFβ-binding domain comprises a fragment of TGFβRI comprising SEQ ID NO: 18. In certain aspects, the TGFβ-binding domain comprises a fragment of TGFβRI consisting of SEQ ID NO: 18.

TABLE 3
TGFβ-binding domains

LQCFCHLCTKDNFTCVTDGLCFVSVTETTDKVIHNSMCIAEIDLIPRDRP
FVCAPSSKTGSVTTTYCCNQDHCNKIELPTTVKSSPGLGPVEL
(SEQ ID NO: 18)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPDLLLVIFQ
(SEQ ID NO: 19)

In some aspects, the TGFβ-binding domain comprises an extracellular domain of TGFβRI, or a TGFβ-binding fragment thereof, comprising one or more point mutation relative to SEQ ID NO: 1, wherein the one or more point mutation increases the affinity of the TGFβ-binding domain for TGFβ as compared to endogenous TGFβRI.

In some aspects, the TGFβ-binding domain comprises the extracellular domain of TGFβRII. In some aspects, the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6. In some aspects, the TGFβ-binding domain comprises the amino acid sequence set forth in SEQ ID NO: 6. In some aspects, the TGFβ-binding domain comprises a fragment of TGFβRII, wherein the fragment is capable of binding TGFβ. In some aspects, the TGFβ-binding domain comprises a fragment of TGFβRII comprising amino acids 23-166 of SEQ ID NO: 4. In some aspects, the TGFβ-binding domain comprises a fragment of TGFβRII comprising at least amino acids D55 and E142 of SEQ ID NO: 4. In some aspects, the TGFβ-binding domain comprises a fragment of TGFβRII comprising amino acids 24-166, amino acids 25-166, amino acids 30-166, amino acids 35-166, amino acids 40-166, amino acids 45-166, amino acids 50-166, amino acids 51-166, amino acids 52-166, amino acids 53-166, amino acids 54-166, or amino acids 55-166 of SEQ ID NO: 4. In some aspects, the TGFβ-binding domain comprises a fragment of TGFβRII comprising amino acids 25-165, amino acids 30-160, amino acids 35-155, amino acids 40-150, amino acids 45-150, amino acids 50-145, amino acids 51-144, amino acids 52-143, amino acids 53-142, amino acids 54-142, or amino acids 55-142 of SEQ ID NO: 4. In some aspects, the TGFβ-binding domain comprises a fragment of TGFβRII comprising amino acids 24-165, amino acids 24-160, amino acids 24-155, amino acids 24-150, amino acids 24-145, amino acids 24-144, amino acids 24-143, or amino acids 24-142 of SEQ ID NO: 4. In certain aspects, the TGFβ-binding domain comprises the amino acid sequence set forth in SEQ ID NO: 19. In certain aspects, the TGFβ-binding domain comprises a fragment of TGFβRII comprising SEQ ID NO: 19. In certain aspects, the TGFβ-binding domain comprises a fragment of TGFβRII consisting of SEQ ID NO: 19.

In some aspects, the TGFβ-binding domain comprises an extracellular domain of TGFβRII, or a TGFβ-binding fragment thereof, comprising one or more point mutation relative to SEQ ID NO: 4, wherein the one or more point mutation increases the affinity of the TGFβ-binding domain for TGFβ as compared to endogenous TGFβRII.

In some aspects, the TGFβ-binding domain comprises an antigen-binding fragment of an anti-TGFβ antibody. An antigen-binding fragment of any anti-TGFβ antibody can be used in the compositions and methods disclosed herein. In some aspects, the TGFβ-binding domain comprises an antigen-binding fragment of fresolimumab (GC1008). In some aspects, the TGFβ-binding domain comprises an antigen-binding fragment of a TGF-β1-specific, humanized, neutralizing mAb (TGF-β1 mAb). See, e.g., Voelker et al., JASN 28(3):953-62 (2017), which is incorporated by reference herein in its entirety.

In some aspects, the chimeric activation receptor comprises a linker between the TGFβ-binding domain and the transmembrane domain. In some aspects, the linker is a flexible linker. In some aspects, the linker is a rigid linker. In some aspects, the linker is a peptide linker. In some aspect, the linker comprises at least about 1 amino acid, at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 25 amino acids, or at least about 30 amino acids. In some aspects, the linker does not comprise a fragment of a human TGFβ receptor. In some aspects, the linker is cleavable.

In certain aspects, the chimeric activation receptor does not comprise a linker.

II.A.2. Transmembrane Domain

In some aspects, the chimeric activation receptor disclosed herein comprises (i) a TGFβ-binding domain, (ii) a transmembrane domain, and (iii) a CD2 costimulatory domain. In some aspects, the transmembrane domain is positioned between the TGFβ-binding domain and the CD2 costimulatory domain. Any transmembrane domain known in the art can be used in the chimeric activation receptors. In some aspects, the transmembrane domain is artificial, e.g., an engineered transmembrane domain. In some aspects, the transmembrane domain is derived from a naturally occurring polypeptide. In some aspects, the transmembrane domain comprises a transmembrane domain from a naturally occurring polypeptide.

In certain aspects, the transmembrane domain comprises a human TGFβRI transmembrane domain or a fragment thereof. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 2. In certain aspects, the transmembrane domain comprises the amino acid sequence set forth in SEQ ID NO: 2. In some aspects, the transmembrane domain comprises amino acids 127-147 of SEQ ID NO: 1.

In certain aspects, the transmembrane domain comprises a human TGFβRII transmembrane domain or a fragment thereof. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5. In certain aspects, the transmembrane domain comprises the amino acid sequence set forth in SEQ ID NO: 5. In some aspects, the transmembrane domain comprises amino acids 167-187 of SEQ ID NO: 4.

In some aspects, the transmembrane domain comprises a CD8 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the transmembrane domain comprises the amino acid sequence set forth in SEQ ID NO: 8.

TABLE 4
Transmembrane Domains.

| | |
|---|---|
| CD8 TM | IYIWAPLAGTCGVLLLSLVITLYC |
| Domain | (SEQ ID NO: 8) |
| CD2 TM | IYLIIGICGGGSLLMVFVALLVFYIT |
| Domain | (SEQ ID NO: 9) |
| CD28 TM | CPSPLFPGPSKPFWVLVVVGGVLACYSLLVTVAFIIFWV |
| Domain | (SEQ ID NO: 20) |
| OX40 TM | VAAILGLGLVLGLLGPLAILL |
| Domain | (SEQ ID NO: 21) |

In some aspects, the transmembrane domain comprises a CD2 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9. In some aspects, the transmembrane domain comprises the amino acid sequence set forth in SEQ ID NO: 9. In some aspects, the transmembrane domain comprises a PD1, TGFbR2, CD4, ICOS, CTLA4, or a DAP10 transmembrane domain.

In some aspects, the transmembrane domain comprises a CD28 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the transmembrane domain comprises the amino acid sequence set forth in SEQ ID NO: 20.

In some aspects, the transmembrane domain comprises an OX40 transmembrane domain. In some aspects, the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the transmembrane domain comprises the amino acid sequence set forth in SEQ ID NO: 21.

In some aspects, the transmembrane domain comprises a CD28 transmembrane domain. In some aspects, the transmembrane domain comprises a CD4 transmembrane domain. In some aspects, the transmembrane domain comprises a PD1 transmembrane domain. In some aspects, the transmembrane domain comprises a TGFbR2 transmembrane domain. In some aspects, the transmembrane domain comprises an OX40 transmembrane domain. In some aspects, the transmembrane domain comprises a CD4 transmembrane domain. In some aspects, the transmembrane domain comprises an ICOS transmembrane domain. In some aspects, the transmembrane domain comprises a CTLA4 transmembrane domain. In some aspects, the transmembrane domain comprises a DAP10 transmembrane domain.

II.A.3. Costimulatory Domain

The chimeric activation receptors disclosed herein comprise at least (i) a TGFβ-binding domain and (ii) a CD2 costimulatory domain. In certain aspects, the CD2 costimulatory domain is a human CD2 costimulatory domain. In some aspects, the CD2 costimulatory domain comprises an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7. In some aspects, the CD2 costimulatory domain comprises the amino acid sequence set forth in SEQ ID NO: 7.

In some aspects, the chimeric activation receptor comprises (i) a TGFβ-binding domain, (ii) a transmembrane domain, and (iii) a CD2 costimulatory domain. In certain aspects, the chimeric activation receptor further comprises a linker between the transmembrane domain and the CD2 costimulatory domain. In some aspects, the linker is a flexible linker. In some aspects, the linker is a rigid linker. In some aspects, the linker is a peptide linker. In some aspect, the linker comprises at least about 1 amino acid, at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 11 amino acids, at least about 12 amino acids, at least about 13 amino acids, at least about 14 amino acids, at least about 15 amino acids, at least about 16 amino acids, at least about 17 amino acids, at least about 18 amino acids, at least about 19 amino acids, at least about 20 amino acids, at least about 25 amino acids, or at least about 30 amino acids. In some aspects, the linker does not comprise a fragment of a human TGFβ receptor. In some aspects, the linker does not comprise a fragment of a human CD2. In some aspects, the linker does not comprise a fragment of a human CD8. In some aspects, the linker is cleavable

II.B. Nucleic Acid Molecules

Certain aspects of the present disclosure are directed to a nucleic acid molecule encoding a chimeric activation receptor comprising (i) a transforming growth factor β (TGFβ)-binding domain; (ii) a transmembrane domain; (iii) and a CD2 costimulatory domain. The chimeric antigen receptor encoded by the nucleic acid molecule can be any chimeric activation receptor disclosed herein.

In some aspects, the nucleic acid molecule further encodes a chimeric antigen receptor (CAR). In some aspects, the nucleic acid molecule further encodes a T cell receptor (TCR). As such, in some aspects, the nucleic acid molecule comprises (i) a portion encoding the chimeric activation receptor and (ii) a portion encoding either (a) a CAR or (b) a TCR. In certain aspects, the portion of the nucleic acid molecule encoding the chimeric activation receptor and the portion of the nucleic acid molecule encoding the CAR or TCR are expressed under the control of a single promoter. In some aspects, the chimeric activation receptor and the CAR or TCR are expressed as a single polypeptide. In some aspects, the portion of the nucleic acid molecule encoding the chimeric activation receptor is expressed under the control of a first promoter and the portion of the nucleic acid molecule encoding the CAR or TCR is expressed under the control of a second promoter. In some aspects, the chimeric activation receptor is expressed from the nucleic acid as a first polypeptide and the CAR or TCR is expressed from the nucleic acid as a second polypeptide.

In some aspects, the portion of the nucleic acid molecule encoding the chimeric activation receptor and the portion of the nucleic acid molecule encoding the CAR or TCR are connected by portion of the nucleic acid encoding a linker. In some aspects, the chimeric activation receptor, the CAR or TCR, and the linker are expressed as a single polypeptide. In some aspects, the linker is a cleavable linker. In some aspects, the linker is selected from a P2A linker, a T2A linker, an F2A linker, an E2A linker, a furin cleavage site, or any combination thereof. In certain aspects, the linker comprises a P2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 11. In some aspects, the linker comprises the amino acid sequence set forth in SEQ ID NO: 11. In some aspects, the P2A linker further comprises a GSG linker sequence.

TABLE 5
Linker Sequences

| | |
|---|---|
| P2A | ATNFSLLKQAGDVEENPGP |
| | (SEQ ID NO: 11) |
| T2A | EGRGSLLTCGDVEENPGP |
| | (SEQ ID NO: 12) |
| F2A | VKQTLNFDLLKLAGDVESNPGP |
| | (SEQ ID NO: 13) |
| E2A | QCTNYALLKLAGDVESNPGP |
| | (SEQ ID NO: 14) |
| Furin Cleavage Site | RAKR |
| | (SEQ ID NO: 15) |

In certain aspects, the linker comprises a T2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 12. In some aspects, the linker comprises the amino acid sequence set forth in SEQ ID NO: 12.

In certain aspects, the linker comprises an F2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 13. In some aspects, the linker comprises the amino acid sequence set forth in SEQ ID NO: 13.

In certain aspects, the linker comprises an E2A linker. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 14. In some aspects, the linker comprises the amino acid sequence set forth in SEQ ID NO: 14.

In certain aspects, the linker comprises an amino acid sequence comprising a furin cleavage site. In some aspects, the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 15. In some aspects, the linker comprises the amino acid sequence set forth in SEQ ID NO: 15.

In other aspects, the nucleic acid molecule comprises (i) a portion encoding a chimeric activation receptor disclosed herein, (ii) an internal ribosome entry site (IRES), and (iii) a portion encoding a CAR or TCR. In certain aspects, the IRES is located in between the portion of the nucleic acid encoding the chimeric activation receptor and the portion of the nucleic acid encoding the CAR or TCR.

II.B.1. Chimeric Antigen Receptors/T Cell Receptors

In certain aspects, the nucleic acid molecule encodes (i) a chimeric activation receptor disclosed herein and (ii) a CAR or a TCR. The CAR or TCR encoded by the nucleic acid molecule can be any CAR or TCR known in the art.

In some aspects, the CAR specifically binds (i.e., target) one or more antigens expressed on a tumor cell, such as a malignant B cell, a malignant T cell, or a malignant plasma cell.

In some aspects, the CAR specifically binds to (i.e., targets) an antigen selected from the group consisting of CD19, TRAC, TCRβ, BCMA, CLL-1, CS1, CD38, CD19, TSHR, CD123, CD22, CD30, CD70, CD171, CD33, EGFRvIII, GD2, GD3, In Ag, PSMA, ROR1, ROR2, GPC1, GPC2, FLT3, 717AG-72, CD44v6, CEA, EPCAM, B71-13, IL-13Ra2, mesothelin, IL1Ra, PSCA, PRSS21, VEGFR2, LewisY, CD24, PDGFR-beta, SSEA-4, CD20, folate receptor alpha, ERBB2 (Her2/neu), MUC1, MUC16, EGER, NCAM, prostase, PAP, ELF2M, Ephrin B2, receptor, CAIX, LMP2, gplOO, ber-abl, tyrosinase, EphA2, fucosyl GM1, sLe, GM3, TGS5, HMWMAA, o-acetyl-GD2, folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, Melan A/MART1, Ras mutant, hTERT, sarcoma translocation breakpoints, ML-IAP, ERG (TMPRSS2 ETS fusion gene), N A17, PAX3, androgen receptor, cyclin B1, MYCN, RhoC, TRP-2, CYP1B1, BORIS, SART3, PAX5, OY-TES1 LCR, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

In some aspects, the CAR targets ROR1. In some aspects, the CAR targets GPC2. In some aspects, the CAR targets BCMA. In some aspects, the CAR targets CD147. In some aspects, the CAR targets CD19. In some aspects, the CAR targets GPC3. In some aspects, the CAR targets CD19 and CD22. In some aspects, the CAR targets CD19 and CD28. In some aspects, the CAR targets CD20. In some aspects, the CAR targets CD20 and CD19. In some aspects, the CAR targets CD22. In some aspects, the CAR targets CD30. In some aspects, the CAR targets CEA. In some aspects, the CAR targets DLL3. In some aspects, the CAR targets EGFRvIII. In some aspects, the CAR targets GD2. In some aspects, the CAR targets HER2. In some aspects, the CAR targets IL-1RAP. In some aspects, the CAR targets mesothelin. In some aspects, the CAR targets methothelin. In some aspects, the CAR targets NKG2D. In some aspects, the CAR targets PSMA. In some aspects, the CAR targets TnMUC1.

In certain aspects, the CAR comprises an antigen-binding domain that specifically binds an antigen in complex with an MHC. In some aspects, the CAR comprises an antigen-binding domain from a TCRm, e.g., any TCRm disclosed herein.

in certain aspects, the CAR comprises a costimulatory domain. Any costimulatory domain known in the art can be used in the CARs of the present disclosure. In some aspects, the CAR comprises a costimulatory domain selected from a costimulatory domain from interleukin-2 receptor (IL-2R), interleukin-12 receptor (IL-12R), IL-7 receptor, IL-21 receptor, IL-23 receptor, IL-15 receptor, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, and any combination thereof. In certain aspects, the CAR comprises a 4-1BB/CD137 costimulatory domain. In certain aspects, the CAR comprises an IL-12R costimulatory domain. In certain aspects, the CAR comprises a CD28 costimulatory domain. In certain aspects, the CAR comprises an IL-7 receptor costimulatory domain. In certain aspects, the CAR comprises an IL-21 receptor costimulatory domain. In certain aspects, the CAR comprises an IL23 receptor costimulatory domain. In certain aspects, the CAR comprises an IL-23 costimulatory domain. In certain aspects, the CAR comprises an IL-15 receptor costimulatory domain.

In some aspects, the nucleic acid molecule encodes a chimeric activation receptor and a TCR, e.g., an engineered TCR. As used herein, the term “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of immune cells, e.g., T cells, NK cells, and/or TILs. In some aspects, the TCR specifically binds a neoantigen identified from a cancer patient.

In some aspects, the TCR specifically binds (i.e., target) one or more antigens expressed on a tumor cell, such as a malignant B cell, a malignant T cell, or a malignant plasma cell.

In some aspects, the TCR engineered cells can target main types: shared tumor-associated antigens (shared TAAs) and unique tumor-associated antigens (unique TAAs), or tumor-specific antigens. The former can include, without any limitation, cancer-testis (CT) antigens, overexpressed antigens, and differentiation antigens, while the latter can include, without any limitation, neoantigens and oncoviral antigens. Human papillomavirus (HPV) E6 protein and HPV E7 protein belong to the category of oncoviral antigens.

In some aspects, the TCR engineered cells can target a CT antigen, e.g., melanoma-associated antigen (MAGE) including, but not limited to, MAGE-A1, MAGE-A2, MAGE-A3, MAGE-A4, MAGE-A6, MAGE-A8, MAGE-A9.23, MAGE-A10, and MAGE-A12. In some aspects, the TCR engineered cells can target glycoprotein (gp100), melanoma antigen recognized by T cells (MART-1), and/or tyrosinase, which are mainly found in melanomas and normal melanocytes. In some aspects, the TCR engineered cells can target Wilms tumor 1 (WT1), i.e., one kind of overexpressed antigen that is highly expressed in most acute myeloid leukemia (AML), acute lymphoid leukemia, almost every type of solid tumor and several critical tissues, such as heart tissues. In some aspects, the TCR engineered cells can target mesothelin, another kind of overexpressed antigen that is highly expressed in mesothelioma but is also present on mesothelial cells of several tissues, including trachea.

In some aspects, the TCR can target any neoantigen, which can be formed by random somatic mutations specific to individual tumors. In some aspects, the TCR specifically binds to (i.e., targets) a cancer antigen selected from the group consisting of AFP, Braf, CD19, TRAC, TCRβ, BCMA, CLL-1, CS1, CD38, CD19, TSHR, CD123, CD22, CD30, CD171, CD33, EGFR ii GD2, GD3, Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gp100, ber-abl, tyrosinase, EphA2, fucosyl GM1. GM3, TGS5, HMWMAA, o-acetyl-GD2, folate receptor beta, TEM1/CD248, TEM7R, CLDN6, GPRCSD, CXORF61, CD97, CD179a, ALK Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-LAP, ERG (TMPRSS2 ETS fusion gene), NA17, PAX3, androgen receptor, cyclin Bl, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

In certain aspects, the TCR specifically binds (i.e., targets) hTERT. In some aspects, the TCR specifically binds (i.e., targets) KRAS. In some aspects, the TCR specifically binds (i.e., targets) Braf. In some aspects, the TCR specifically binds (i.e., targets) TGFβRII. In some aspects, the TCR specifically binds (i.e., targets) MAGE A10/A4. In some aspects, the TCR specifically binds (i.e., targets) AFP. In some aspects, the TCR specifically binds (i.e., targets) PRAME. In some aspects, the TCR specifically binds (i.e., targets) MAGE A1. In some aspects, the TCR specifically binds (i.e., targets) WT-1. In some aspects, the TCR specifically binds (i.e., targets) NY-ESO. In some aspects, the TCR specifically binds (i.e., targets) PRAMS. In some aspects, the TCR specifically binds (i.e., targets) NY-ESO. In some aspects, the TCR specifically binds (i.e., targets) CD19. In certain aspects, the TCR specifically binds a neoantigen identified from a cancer patient.

In some aspects, the TCR comprises an intracellular gamma/delta domain. In some aspects, the TCR is an antibody-T-cell receptor (AbTCR) (see, e.g., Xu et al., Cell Discovery 4:62 (2018), which is incorporated by reference herein in its entirety.

In some aspects, the nucleic acid molecule encodes a chimeric activation receptor and a T cell receptor mimic (TCRm), also known as a TCR-like antibody. TCRm are a type of antibody that recognize epitopes comprising both the peptide and the MHC-I molecule, similar to the recognition of such complexes by the TCR on T cells (see, e.g., Traneska et al., Front. Immunol. 8(1001):1-12 (2017), which is incorporated by reference herein in its entirety). In some aspects, the TCRm specifically binds to a tumor antigen. In certain aspects, the TCRm specifically binds a neoantigen identified from a cancer patient.

In some aspects, the TCRm specifically binds (i.e., target) one or more antigens expressed on a tumor cell, such as a malignant B cell, a malignant T cell, or a malignant plasma cell. In some aspects, the TCRm is a monoclonal antibody. In some aspects, the TCRm specifically binds to WT1. In some aspects, the TCRm specifically binds to a fragment of WT1. In some aspects, the TCRm comprises ESK1 (see, e.g., Ataie et al., J. Mol. Biol. 428(1):194-205 (2016), which is incorporated by reference herein in its entirety). In some aspects, the TCRm specifically binds to MAGE-A1. In some aspects, the TCRm specifically binds to p68 RNA helicase/HLA-A*02:01. In some aspects, the TCRm specifically binds to hCG-b/HLAA*02:01. In some aspects, the TCRm specifically binds to Her2-E75/HLA-A*02:01. In some aspects, the TCRm specifically binds to PR-1 in context of HLA-A*02:01 (see, e.g., Oncoimmunology 5 (1):e1049803 (June 2015), which is incorporated by reference herein in its entirety). In some aspects, the TCRm specifically binds to the survivin-2B-derived nonamer peptide, AYACNTSTL (SEQ ID NO: 15; SV2B80-88), presented on FILA-A*24 (SV2B80-88/HLA-A*24) (see, e.g., Kurosawa et al., Nature Scientific Reports 9(9827):1-11 (2019), which is incorporated by reference herein in its entirety). In some aspects, the TCRm specifically binds one or more tumor-associated PRAME peptide/HLA-I antigens (see, e.g., J Clin Invest. 127(7):2705-18 (2017), which is incorporated by reference herein in its entirety). In some aspects, the TCRm specifically binds to tyrosinase. In some aspects, the TCRm specifically binds telomerase catalytic subunit. In some aspects, the TCRm specifically binds to glycoprotein 100 (gp100). In some aspects, the TCRm specifically binds to mucin 1 (MUC1). In some aspects, the TCRm specifically binds to human telomerase reverse transcriptase (hTERT). In some aspects, the TCRm specifically binds to NYESO-1. In some aspects, the TCRm specifically binds to MART-1. In some aspects, the TCRm specifically binds to PRAME.

In some aspects, the TCRm specifically binds to a viral antigen. In some aspects, the TCRm specifically binds to Env183/A2 (Hep B/HLA-A*02:01). In some aspects, the TCRm specifically binds to KP14/1 and KP15/11 (HIV envelope gp160/HLAA*02:01). In some aspects, the TCRm specifically binds to RL36A (West Nile Virus/mouse H-2db). In some aspects, the TCRm specifically binds to a viral epitope derived from HTLV. In some aspects, the TCRm specifically binds to a viral epitope derived from influenza. In some aspects, the TCRm specifically binds to a viral epitope derived from CMV. In some aspects, the TCRm specifically binds to a viral epitope derived from HIV.

II.B.2. Vectors

In some aspects, the present disclosure provides a vector comprising an isolated nucleic acid molecule that encodes a chimeric activation receptor disclosed herein, an isolated nucleic acid molecule that encodes a chimeric activation receptor and a CAR, a nucleic acid molecule that encodes a chimeric activation receptor and a TCR, a nucleic acid molecule that encodes a chimeric activation receptor and a TCRm, or any combination thereof. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In certain aspects, the nucleic acid is a cDNA molecule. Nucleic acids described herein can be obtained using standard molecular biology techniques known in the art.

In some aspects, the disclosure provides a vector comprising a polynucleotide encoding a nucleic acid molecule that encodes a chimeric activation receptor, disclosed herein. In some aspects, the disclosure provides a vector comprising a polynucleotide encoding a nucleic acid molecule that encodes a chimeric activation receptor, disclosed herein, and a CAR. In some aspects, the disclosure provides a vector comprising a polynucleotide encoding a nucleic acid molecule that encodes a chimeric activation receptor, disclosed herein, and a TCR. In some aspects, the disclosure provides a vector comprising a polynucleotide encoding a nucleic acid molecule that encodes a chimeric activation receptor, disclosed herein, and a TCRm.

In some aspects, the present disclosure provides a vector comprising one or more polynucleotides encoding a chimeric activation receptor disclosed herein, operatively linked to a promoter.

In some aspects, the present disclosure provides a vector comprising (a) one or more polynucleotides encoding a chimeric activation receptor disclosed herein, operatively linked to a first promoter; and (b) one or more polynucleotides encoding a CAR, wherein the one or more polynucleotides encoding the CAR are operatively linked to a second promoter.

In some aspects, the present disclosure provides a vector comprising (a) one or more polynucleotides encoding a chimeric activation receptor disclosed herein, operatively linked to a first promoter; and (b) one or more polynucleotides encoding a TCR, wherein the one or more polynucleotides encoding the TCR are operatively linked to a second promoter.

In some aspects, the present disclosure provides a vector comprising (a) one or more polynucleotides encoding a chimeric activation receptor disclosed herein, operatively linked to a first promoter; and (b) one or more polynucleotides encoding a TCRm, wherein the one or more polynucleotides encoding the TCRm are operatively linked to a second promoter.

In some aspects, multiple open reading frames encoding the chimeric activation receptor, the CAR, the TCR, and/or the TCRm are operatively linked to a single promoter (e.g., an inducible promoter). In some aspects, each polynucleotide is operatively linked to a different promoter, i.e., the expression of polypeptide is independently controlled by its own promoter (e.g., an inducible promoter). When multiple inducible promoters are present, they can be induced by the same inducer molecule or a different inducer.

In some aspects, a nucleic acid molecule encoding a chimeric activation receptor disclosed herein (and/or a nucleic acid molecule encoding CAR, TCR, and/or TCRm) can be operably linked to one or more regulatory elements. Regulatory elements can include, e.g., promoters/enhancers (such as exhaustion-responsive promoters, activation-responsive promoters, cytokine-responsive promoters, calcium-responsive promoters, and the like), localization sequences (such as membrane-localization sequences, nuclear localization sequences, nuclear exclusion sequences, proteasomal targeting sequences, and the like), post-translational modification sequences (such as ubiquitination, phosphorylation, dephosphorylation, and the like).

Suitable vectors for the disclosure include expression vectors, viral vectors, and plasmid vectors. In some aspects, the vector is a viral vector, a mammalian vector, or a bacterial vector. In some aspects, the vector is a viral vector. As used herein, the terms “vector” and “expression vector” refers to any nucleic acid construct that contains the necessary elements for the transcription and translation of an inserted coding sequence, or in the case of an RNA viral vector, the necessary elements for replication and translation, when introduced into an appropriate host cell. Expression vectors can include plasmids, linear ssDNA, linear dsDNA, phagemids, viruses, and derivatives thereof.

One can readily employ any vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, the vector is a retroviral vector. As used herein, viral vectors include, but are not limited to, selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector. In some specific aspects, the vector is a lentivirus. Examples of lentiviral vectors are disclosed in International Application Publication Nos. WO9931251, WO9712622, WO9817815, WO9817816, and WO9818934, each of which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA). Other plasmids are well-known to those of ordinary skill in the art. Additionally, plasmids can be custom designed using standard molecular biology techniques to remove and/or add specific fragments of DNA.

In some aspects, the vector comprises a first polynucleotide encoding a chimeric activation receptor of the present disclosure and a second polynucleotide encoding a CAR, TCR, and/or TCRm. Thus, in some aspects, the polynucleotide encoding the chimeric activation receptor polypeptide and the polynucleotide encoding the CAR, TCR, and/or TCRm are on the same vector. In other aspects, the polynucleotide encoding the chimeric activation receptor polypeptide and the second polynucleotide encoding the CAR, TCR, and/or TCRm polypeptide are on different vectors.

In some aspects, the polynucleotides disclosed herein are integrated into the genome of a host cell (e.g., a nucleic acid encoding a chimeric activation receptor, a CAR, a TCR, and/or a TCRm of the present disclosure can be integrated into the genome of an immune cell, e.g., a T-cell).

In some aspects, the vector comprises a transposable element. In certain aspects, the vector comprises a polynucleotide encoding a chimeric activation receptor disclosed herein flanked by at least two transposon-specific inverted terminal repeats (ITRs). In some aspects, the transposon-specific ITRs are recognized by a DNA transposon. In some aspects, the transposon-specific ITRs are recognized by a retrotransposon. Any transposon system known in the art can be used to introduce the nucleic acid molecules into the genome of a host cell, e.g., an immune cell. In some aspects, the transposon is selected from hAT-like Tol2, Sleeping Beauty (SB), Frog Prince, piggyBac (PB), and any combination thereof. In some aspects, the transposon comprises Sleeping Beauty. In some aspects, the transposon comprises piggyBac. See, e.g., Zhao et al., Transl. Lung Cancer Res. 5(1):120-25 (2016), which is incorporated by reference herein in its entirety.

In some aspects, the polynucleotides disclosed herein are DNA (e.g., a DNA molecule or a combination thereof), RNA (e.g., a RNA molecule or a combination thereof), or any combination thereof. The polynucleotides disclosed herein comprise nucleic acid sequences comprising single stranded or double stranded RNA or DNA (e.g., ssDNA or dsDNA) in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure (e.g., a chimeric activation receptor of the present disclosure). The disclosure further provides expression vectors comprising the polynucleotides of the disclosure (e.g., a polynucleotide encoding a chimeric activation receptor of the present disclosure) operatively linked to a promoter, e.g., a constitutively active promoter or an inducible promoter. The disclosure further provides cells comprising the expression vectors comprising polynucleotides of the present disclosure operatively linked to a promoter.

The vectors disclosed herein can comprise a nucleic acid coding region (e.g., a polynucleotide encoding a chimeric activation receptor disclosed herein) operatively linked to any control sequences capable of affecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences of the disclosure, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various aspects, the expression vector may comprise a plasmid, viral-based vector, or any other suitable expression vector as discussed above.

II.C. Cells

Certain aspects of the present disclosure are directed to a cell comprising a chimeric activation receptor disclosed herein. In some aspect, the cell comprises a chimeric activation receptor comprising (i) a TGFβ-binding domain; (ii) a transmembrane domain; (iii) and a CD2 costimulatory domain. In some aspects, the cell further comprises (e.g., expresses) an endogenous TGFβRI. In some aspects, the cell further comprises (e.g., expresses) an endogenous TGFβRII. In some aspects, the cell further comprises (e.g., expresses) an endogenous TGFβRI and an endogenous TGFβRII. In some aspects, the chimeric activation receptor comprises an extracellular domain of TGFβRI, and the cell does not express an endogenous TGFβRI, e.g., the endogenous TGFβRI is mutated or knocked out.

In certain aspects, the cell is an immune cell. In some aspects, the immune cell is isolated from a human subject. In some aspects, the immune cell is isolated from a human subject for allogeneic cell therapy. In some aspects, the immune cell is isolated from a human subject for autologous cell therapy. In some aspects, the cell is a T cell. In some aspects, the T cell is a Th1, Th2, Th17, or Tc17 cell. In some aspects, the cell is an NK cell. In some aspects, the cell is a TIL. In some aspects, the cell is a Treg. In some aspects, the cell is a natural killer T (NKT) cell. In some aspects, the cell is a B cell. In some aspects, the immune cell is a tumor-infiltrating T cell. In some aspects, the immune cell is a tumor-infiltrating NK cell.

In certain aspects, the immune cell is differentiated from a pluripotent or multipotent progenitor cell. As such, as used herein, an “immune cell” further includes a pluripotent or multipotent cell that can give rise to a mature immune cell. In some aspects, the cell (e.g., the immune cell) is an induced pluripotent stem cell (IPSC). In some aspects, the cell (e.g., the immune cell) is an embryonic stem cell. In some aspects, the cell is a hematopoietic stem cell.

In some aspects, the T cell is a CD4⁺ T cell. In some aspects, the T cell is a CD8⁺ T cell. In some aspects, the T cell is a naïve T (T_{N}) cell. In some aspects, the T cell is CD95⁻/CD45RA⁺/CD62L⁺/CCR7⁺. In some aspects, the T cell is CD95⁺/CD45RA⁺/CD62L⁺/CCR7⁺. In some aspects, the T cell is CD45RO⁺/CCR7⁺/CD62L⁺. In some aspects, the T cell is CD45RO⁺/CCR7⁻/CD62L⁻.

In certain aspects, the cell, e.g., immune cell, is further engineered to comprise a CAR. In certain aspects, the cell, e.g., the immune cell, comprises (i) a chimeric activation receptor and (ii) a CAR. The cell can be engineered to express the chimeric activation receptor and the CAR from a single nucleic acid molecule, e.g., any nucleic acid molecule disclosed herein. Alternatively, a cell, e.g., an immune cell, can be modified to express a chimeric activation receptor disclosed herein from a first nucleic acid molecule and a CAR from a second nucleic acid molecule. Any CAR known in the art can be used in the cells of the present disclosure. In certain aspects, the CAR is selected from any CAR disclosed in section II.B.1., above. In certain aspects, the CAR specifically binds ROR1. In some aspects, the CAR specifically binds GPC2. In some aspects, a CAR-expressing cell is a CAR T cell, e.g., a mono CAR T cell, a genome-edited CAR T cell, a dual CAR T cell, or a tandem CAR T cell. Examples of such CAR T cells are provided in International Application No. PCT/US2019/044195.

In certain aspects, the cell, e.g., immune cell, is further engineered to comprise a TCR. In certain aspects, the cell, e.g., the immune cell, comprises (i) a chimeric activation receptor and (ii) a TCR. The cell can be engineered to express the chimeric activation receptor and the TCR from a single nucleic acid molecule, e.g., any nucleic acid molecule disclosed herein. Alternatively, a cell, e.g., an immune cell, can be modified to express a chimeric activation receptor disclosed herein from a first nucleic acid molecule and a TCR from a second nucleic acid molecule. Any TCR known in the art can be used in the cells of the present disclosure. In certain aspects, the TCR is selected from any TCR disclosed in section II.B.1., above.

In certain aspects, the cell, e.g., immune cell, is further engineered to comprise a TCRm. In certain aspects, the cell, e.g., the immune cell, comprises (i) a chimeric activation receptor and (ii) a TCRm. The cell can be engineered to express the chimeric activation receptor and the TCRm from a single nucleic acid molecule, e.g., any nucleic acid molecule disclosed herein. Alternatively, a cell, e.g., an immune cell, can be modified to express a chimeric activation receptor disclosed herein from a first nucleic acid molecule and a TCRm from a second nucleic acid molecule. Any TCRm known in the art can be used in the cells of the present disclosure. In certain aspects, the TCRm is selected from any TCRm disclosed in section II.B.1., above.

Certain aspects of the present disclosure are directed to methods of making a chimeric activation receptor disclosed herein by transfecting a cell, e.g., an immune cell, with a nucleic acid molecule disclosed herein and culturing the cell under suitable conditions.

II.D. Pharmaceutical Compositions

The present disclosure also provides a composition comprising a polynucleotide encoding a chimeric activation receptor of the present disclosure, a nucleic acid molecule encoding a chimeric activation receptor and a CAR or TCR of the present disclosure, a vector comprising a nucleic acid molecule of the present disclosure, or a cell expressing a chimeric activation receptor disclosed herein. In some aspects, the composition is used for treating a subject in need of a CAR therapy.

In some aspects, the composition is a pharmaceutical composition. Accordingly, the present disclosure provides, e.g., pharmaceutical compositions comprising (i) a cell, e.g., an immune cell, that has been modified to express a chimeric activation receptor disclosed herein, or (ii) a cell, e.g., an immune cell, that has been modified to express a chimeric activation receptor disclosed herein and a CAR or TCR; and a pharmaceutically acceptable carrier, excipient, or stabilizer. As described herein, such pharmaceutical compositions can be used to prevent and/or treat a cancer. In some aspects, an immune cell of the present disclosure (i.e., a cell expressing a chimeric activation receptor disclosed herein), present in a pharmaceutical composition disclosed herein is a T cell or an NK cell.

As used herein, the term “pharmaceutical composition” refers to one or more of the compounds described herein, such as, e.g., a chimeric activation receptor of the present disclosure (e.g., a nucleic acid molecule encoding the chimeric activation receptor, a vector comprising a nucleic acid molecule encoding the chimeric activation receptor, or a chimeric activation receptor polypeptide) or a cell expressing a chimeric activation receptor of the present disclosure, mixed or intermingled with, or suspended in one or more other chemical components, such as pharmaceutically-acceptable carriers and excipients. One purpose of a pharmaceutical composition is to facilitate administration of preparations of, e.g., cell expressing a chimeric activation receptor of the present disclosure to a subject.

The terms “excipient” and “carrier” are used interchangeably and refer to an inert substance added to a pharmaceutical composition to further facilitate administration of a compound, e.g., a chimeric activation receptor of the present disclosure.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricularly, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The compositions to be used for in vivo administration can be sterile. This is readily accomplished by filtration through, e.g., sterile filtration membranes.

### III. Methods of the Disclosure

Certain aspects of the present disclosure are directed to methods of administering a cell of the disclosure, e.g., an immune cell expressing a chimeric activation receptor, to a subject in need thereof. Certain aspects of the present disclosure are directed to methods of treating a disease of condition in a subject in need thereof, comprising administering to the subject a cell disclosed herein e.g., an immune cell comprising a chimeric activation receptor. In some aspects, the disease or condition comprises a tumor, i.e., a cancer. In some aspects, the method comprises stimulating a T cell-mediated immune response to a target cell population or tissue in a subject, comprising administering an effective amount of a cell composition of the disclosure, e.g., an immune cell comprising a chimeric activation receptor disclosed herein. In some aspects, the target cell population comprises a tumor. In some aspects, the tumor is a solid tumor.

In some aspects, the tumor microenvironment in the subject comprises one or more cells that express TGFβ. In some aspects, one or more tumor cells in the subject expresses TGFβ. In some aspects, one or more fibroblasts, MDSC-myeloid derived suppressor cells, Treg, macrophages, or any combination thereof in the tumor microenvironment express TGFβ. In some aspects, the level of TGFβ in the tumor microenvironment is at least about 25%, at least about %, at least about 50%, at least about 75%, at least about 100%, at least about 125%, at least about 150%, at least about 175%, at least about 200%, at least about 250%, at least about 300%, at least about 350%, at least about 400%, at least about 450%, at least about 500%, at least about 600%, at least about 700%, at least about 800%, at least about 900%, or at least about 1000% higher than the level of TGFβ in a healthy tissue.

In some aspects, administering the cell composition of the disclosure (e.g., an immune cell expressing a chimeric activation receptor) reduces a tumor volume in the subject compared to a reference tumor volume. In some aspects, the reference tumor volume is the tumor volume in the subject prior to the administration of the cell. In further aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In certain aspects, administering the cell composition of the disclosure (e.g., an immune cell expressing a chimeric activation receptor) can reduce the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration of the cell composition of the disclosure. In further aspects, the reference tumor weight is the tumor weight in a corresponding subject that did not receive the administration.

In some aspects, administering the cell composition of the disclosure (e.g., an immune cell expressing a chimeric activation receptor) to a subject, e.g., suffering from a tumor, can increase the number and/or percentage of TILs (e.g., CD4⁺ or CD8⁺) in a tumor and/or a tumor microenvironment (TME) of the subject. In certain aspects, the number and/or percentage of TILs in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the cell composition of the present disclosure or the same subject prior to the administration of the cell composition of the present disclosure).

In some aspects, administering the cell composition of the disclosure (e.g., an immune cell expressing a chimeric activation receptor) to a subject, e.g., suffering from a tumor, can increase the duration of an immune response in a subject relative to the duration of an immune response in a subject administered a similar cell therapy comprising cells lacking a chimeric activation receptor disclosed herein. In some aspects, administering the cell composition of the disclosure (e.g., an immune cell expressing a chimeric activation receptor) to a subject, e.g., suffering from a tumor, can increase the duration of an immune response in a subject relative to the duration of an immune response in a subject administered a similar cell therapy comprising cells comprising a TGFβRII-binding domain fused to a CD28 costimulatory domain. In certain aspects, the duration of the immune response is increased by at least about 10%, at least about 20%, at least about 30%, at least about 40%, at least about 50%, at least about 75%, at least about 100%, at least about 150%, at least about 200%, at least about 300%, at least about 400%, at least about 500%, or at least about 1000% or more compared to a reference (e.g., a subject administered a similar cell therapy comprising cells lacking a chimeric activation receptor disclosed herein). In certain aspects, the duration of the immune response is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., a subject administered a similar cell therapy comprising cells lacking a chimeric activation receptor disclosed herein).

In addition to the above, administering the cell composition of the disclosure (e.g., an immune cell expressing a chimeric activation receptor) can have other effects which are conducive for the treatment of a tumor.

As described herein, the cell composition of the disclosure (e.g., an immune cell expressing a chimeric activation receptor) is used to treat variety of cancer types, e.g., a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

In some aspects, the cell composition of the disclosure (e.g., an immune cell expressing a chimeric activation receptor) is used in combination with other therapeutic agents (e.g., anti-cancer agents and/or immunomodulating agents). Accordingly, in certain aspects, a method of treating a tumor disclosed herein comprises administering the cell composition of the disclosure in combination with one or more additional therapeutic agents.

In some aspects, the cell composition of the disclosure (e.g., an immune cell expressing a chimeric activation receptor) is administered to the subject prior to or after the administration of the additional therapeutic agent. In other aspects, the cell composition of the disclosure (e.g., an immune cell expressing a chimeric activation receptor) is administered to the subject concurrently with the additional therapeutic agent. In certain aspects, the cell composition of the disclosure (e.g., an immune cell expressing a chimeric activation receptor) and the additional therapeutic agent can be administered concurrently as a single composition in a pharmaceutically acceptable carrier. In other aspects, the cell composition of the disclosure (e.g., an immune cell expressing a chimeric activation receptor) and the additional therapeutic agent are administered concurrently as separate compositions.

In some aspects, the subject is a nonhuman animal such as a rat or a mouse. In some aspects, the subject is a human.

In some aspects, a cell composition disclosed herein (e.g., an immune cell expressing a chimeric activation receptor) is used in combination with other therapeutic agents (e.g., anti-cancer agents and/or immunomodulating agents). Accordingly, in certain aspects, a method of treating a tumor disclosed herein comprises administering a cell composition of the present disclosure (e.g., an immune cell expressing a chimeric activation receptor) in combination with one or more additional therapeutic agents to a subject. Such agents can include, for example, chemotherapeutic drug, targeted anti-cancer therapy, oncolytic drug, cytotoxic agent, immune-based therapy, cytokine, surgery, radiotherapy, activator of a costimulatory molecule, immune checkpoint inhibitor, a vaccine, a cellular immunotherapy, or any combination thereof.

In some aspects, a cell composition disclosed herein (e.g., an immune cell expressing a chimeric activation receptor) is used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, a cell composition of the present disclosure (e.g., an immune cell expressing a chimeric activation receptor) is used in combination with one or more anti-cancer agents, such that multiple elements of the immune pathway can be targeted. Non-limiting examples of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically engineered cells, e.g., cells engineered to express a chimeric antigen receptor (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab (OPDIVO®), pembrolizumab (KEYTRUIDA®; MK-3475), pidilizumab (CT-011), PDR001, MEDI0680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRIQ®; RG7446; MPDL3280A; R05541267), durvalumab (MEDI4736, IMFINZI®), BMS-936559, avelumab (BAVENCIO®), LY3300054, CX-072 (Proclaim-CX-072), FAZ053, KN035, MDX-1105, and combinations thereof); and anti-CTLA-4 antibody (e.g., ipilimumab (YERVOY®), tremelimumab (ticilimumab; CP-675,206), AGEN-1884, ATOR-1015, and combinations thereof).

In some aspects, an anti-cancer agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), TIM3 agonist (e.g., anti-TIM3 antibody), or combinations thereof.

In some aspects, a cell composition disclosed herein (e.g., an immune cell expressing a chimeric activation receptor) is administered to the subject prior to or after the administration of the additional therapeutic agent. In other aspects, cell composition disclosed herein (e.g., an immune cell expressing a chimeric activation receptor) is administered to the subject concurrently with the additional therapeutic agent. In certain aspects, the cell composition disclosed herein (e.g., an immune cell expressing a chimeric activation receptor) and the additional therapeutic agent can be administered concurrently as a single composition in a pharmaceutically acceptable carrier. In other aspects, the cell composition disclosed herein (e.g., an immune cell expressing a chimeric activation receptor) and the additional therapeutic agent are administered concurrently as separate compositions. In some aspects, the additional therapeutic agent and the cell composition disclosed herein (e.g., an immune cell expressing a chimeric activation receptor) are administered sequentially.

Some aspects are directed to methods of modulating TGFβ activity in a tumor microenvironment comprising administering a cell disclosed herein, e.g., an immune cell expressing a chimeric activation receptor, to a subject. In some aspects, the modulation comprises converting an endogenous TFGβ signal that inhibits an immune response into a signal that enhances an immune response.

### EXAMPLES

### Example 1— T Cells Expressing a TGFβ-DNR Signal Convertor and a Chimeric Antigen Receptor (CAR)

Illustrative TGFβR2-DNR chimeric receptor constructs were designed as shown in FIG. 2. Optimal T cell costimulatory signaling is initiated by the ligation to the corresponding ligands (CD2, CD28 and OX40). To convert immunosuppressive TGFβ signal to induce costimulatory signaling after exposure to TGFβ, the intracellular domain of TGFβR2 was replaced with T cell costimulatory signaling domains (CD2, CD28 and OX40). The TGFβR2 extracellular domain was connected to T cell costimulatory (CD2) signaling domains with a CD8a transmembrane domain, CD28 and OX40 costimulatory domains were connected to TGFβR2 extracellular domain with their endogenous transmembrane domains respectively.

Expression Constructs: Lentiviral constructs were generated with bi-cistronic expression cassettes. The coding sequences for (i) a ROR1-specific R12 CAR, (ii) a P2A self-cleaving peptide, and (iii) TGFβR2-DNR-chimeric receptors were linked in frame and placed under the control of an MND promoter. The R12 CAR was derived from the R12 anti-ROR1 antibody (Yang et al., PLoS One. (2011) 6:e21018) and contained an appropriate space between (e.g., one of the indicated spacers) the anti-ROR1 antibody and the transmembrane domain, a CD28-derived transmembrane domain, a 4-1BB costimulatory domain, and a CD3 zeta signaling domain.

### Example 2— CAR T Cells Expressing TGFβR2-DNR-Chimeric Receptors are Resistant to TGFβ Immunosuppressive Signals

Assays were performed to determine the effects of TGFβR2-DNR chimeric receptor with various costimulatory domains (CD2, CD28 and OX40) driven by TGFβ on inducing ROR1-directed cytokine release and potency by T cells expressing R12 CAR in the presence of TGFβ.

Methods: Cell Culture and Lentiviral Transduction: Pre-selected, cryopreserved primary human CD4+ and CD8+ T cells from normal donors were obtained from Bloodworks (Seattle WA). Human T cells were cultured in OpTmizer medium (Thermo Fisher) supplemented with Immune Cell Serum Replacement (Thermo Fisher), 2 mM L-glutamine (Gibco), 2 mM Glutamax (Gibco), 200 ‘Um’ IL-2 (R&D systems), 120 IU/ml IL-7 (R&D systems), and 20 IU/ml IL-15 (R&D systems). For lentiviral transduction, the T cells were stimulated with a 1:100 dilution of T cell TransAct (Miltenyi) for 30 hours. Virus was then added to the T cells for 24 hours. Stimulation and viral infection were then terminated by addition of 7 volumes of fresh media without TransAct, and cells were cultured for 7 additional days in Grex-24 plate (Wilson Wolf) prior to cryopreservation in CryoStor CS10 (STEMCELL Technologies) at 3×10⁷ cells/ml. All freshly thawed T cells were normalized for % CAR+ and total T cells by adding Mock (untransduced) T cells to appropriate samples prior to assay set-up.

CAR expression measurement via flow cytometry: To measure CAR expression levels and transduction efficiencies, roughly 0.5×10⁶ cells were pelleted after a 6-day production period following lentiviral transduction for flow cytometry analysis. Cells were resuspended in Fixable Viability Dye eFluor 780 (Invitrogen, Cat #65-0865-14) in PBS for 10 minutes, then washed with Cell Staining Buffer (BioLegend, Cat #420201).

R12 CARs scFv surface expression was detected using recombinant human ROR1-Fc chimera protein (R&D, Cat #9490-RO-050) prelabeled with DyLight™ 650 Microscale Antibody Labeling Kit (ThermoFisher, Cat #84536) diluted 1:500 in Cell Staining Buffer. TGFβR2 surface expression was determined by surface staining of anti-TGFβR2 (R & D systems, Cat #FAB2411P) diluted 1:50 in Cell Staining Buffer, Cells were pelleted after a 20 minutes incubation in the dark, followed by 2×wash with Cell Staining Buffer. FASER Kit was used to amplify the fluorescence intensity (Miltenyi, 130-091-764) according to the manufacturer protocol. All flow cytometric analysis was done on ATTUNE (Life Technologies) and analyzed with FlowJo (Tree Star).

Assay set-up—cell proliferation assay: Transduced primary T cells expressing R12 CAR with TGFβR2-DNR chimeric receptors were counted and resuspended to a cell density equal to 0.4×10⁶ CAR+ cells/ml in cell-assay media, and a 1 ml volume were added to a 12 well plate containing 0.4×10⁶ H1975-NLR cells in 1 ml cell-assay media and incubated at 37 C +/− human recombinant TGFβ (10 ng/ml). No exogenous IL-2 was used for support in this assay. ROR1 CAR cells were counted at day 7 to measure cell proliferation and CAR %.

Assay set-up—target-dependent cytokine secretion: Transduced primary T cells expressing R12 CAR with TGFβR2-DNR chimeric receptors were counted and resuspended to a cell density equal to 0.5×10⁶ CAR+ cells/ml in cell-assay media, and a 100 μl volume, or 50,000 CAR+ cells, were added to a flat-bottom 96 well plate containing 50,000 ROR1+H1975 and A549 target cells and incubated at 37° C. in an IncuCyte Live Cell Analysis System (Sartorius). After 24 hours, supernatant from each well was collected for IL-2 and IFNg measurement according to the manufacturer's protocol (Meso Scale Discovery).

Assay set-up— serial killing: Followed by the coculture set up as described in target-dependent cytokine secretion assay. Each well was imaged every 6 hours in an IncuCyte Live Cell Analysis System (Sartorius) for quantifying the number of H1975 and A549 cells to assess the kinetics of T cell cytotoxicity. Target cell lysis was tracked over 4 days. Every 3-4 days, one fourth of the content was transferred from the previous co-culture plate to a new 96-well plate containing 50,000 fresh A549 or H1975 cells in 200 ul cell-assay media +/− TGFβ. The numbers of remaining H1975 and A549 cells over the 4 rounds of co-culture with R12 CART cells were tracked and normalized to the first time point of each round.

Assay set-up—serial stimulation assay: Transduced primary T cells expressing R12 CAR with TGFβR2-DNR chimeric receptors were counted and resuspended to a cell density equal to 0.4×10⁶ CAR+ cells/ml in cell-assay media, and a 1 ml volume were added to a 12 well plate containing 0.4×10⁶ H1975-NLR cells in 1 ml cell-assay media and incubated at 37° C. +/− human recombinant TGFβ (long/ml). No exogenous IL-2 was used for support in this assay. ROR1 CAR cells were counted and phenotyped every 7 days to measure cell proliferation and CAR %. ROR1 CAR cells coculture with ROR1+ target cells were reset in 1:1 E to T ratio every 7 days. Cytokine production (IFNg and IL2) of ROR1 CAR cells was measured 24 hr after coculture set up each round by MSD.

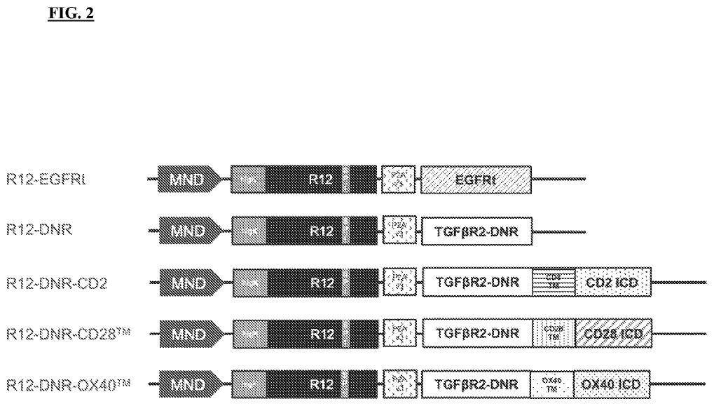

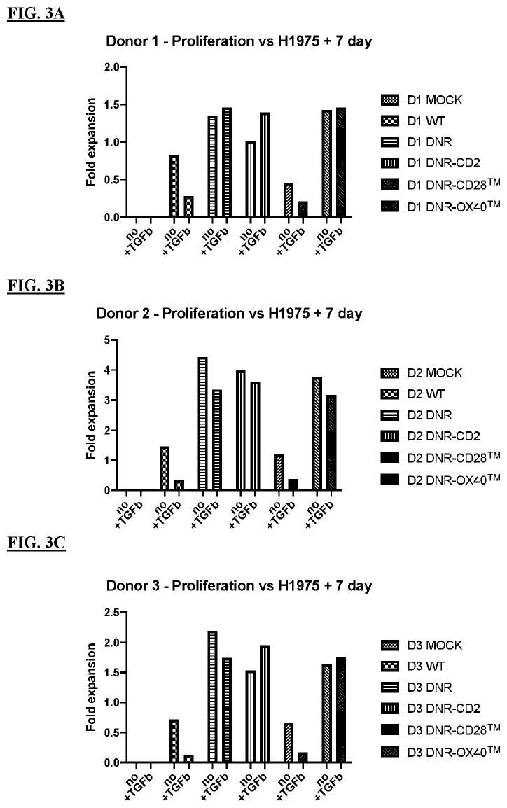

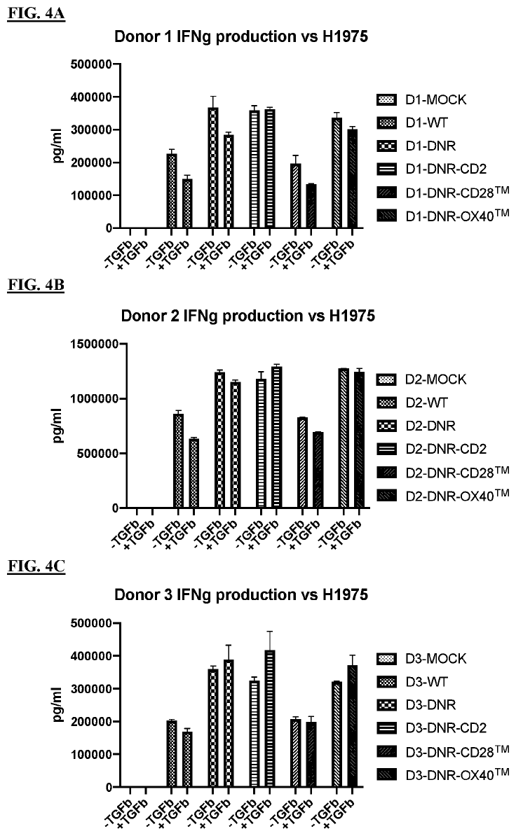

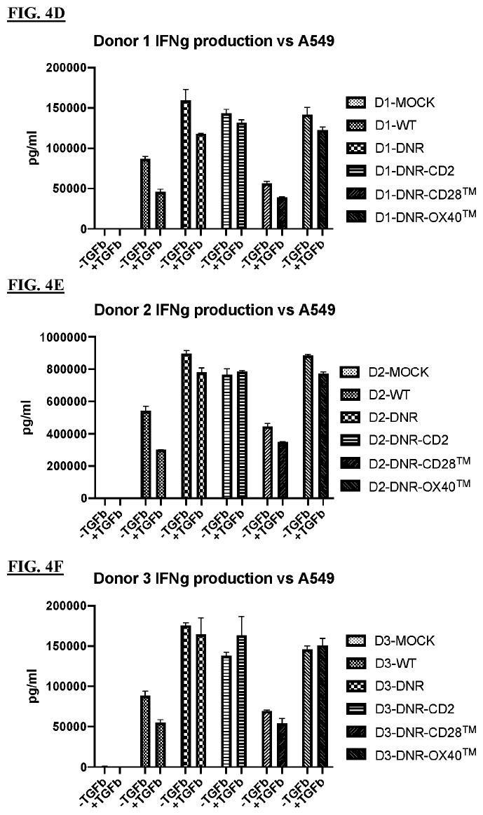

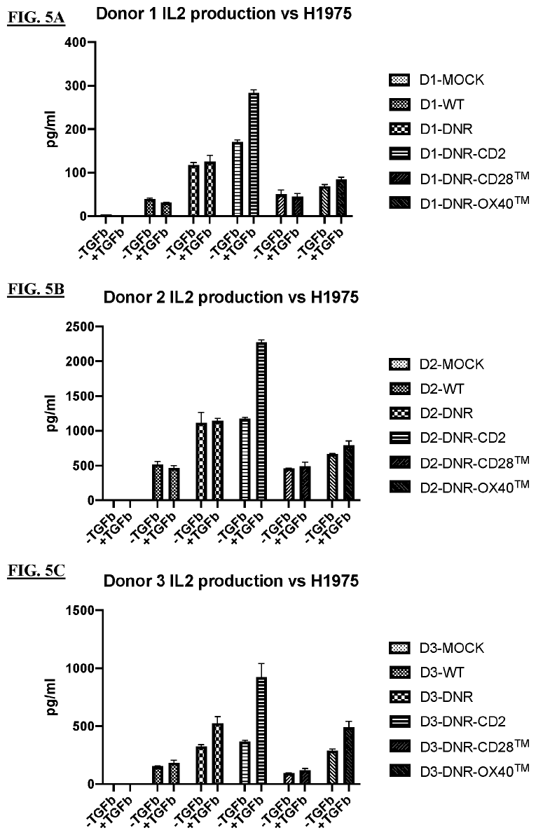

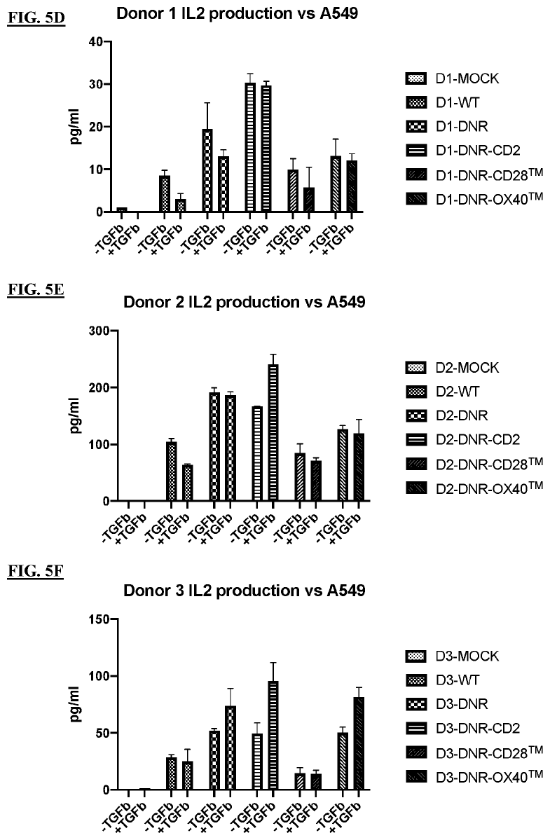

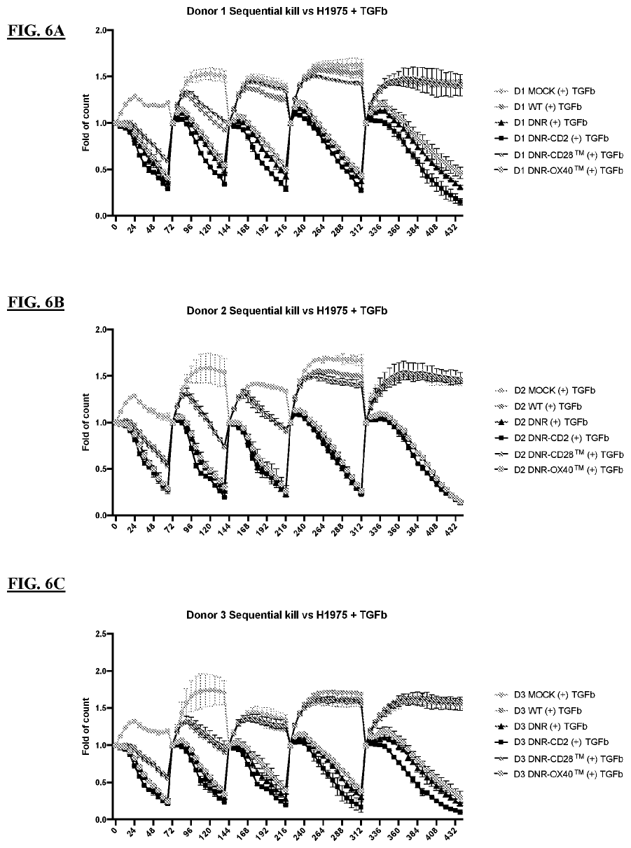

Results: TGFβ signaling decreases T cell expansion and IFNg production in response to antigen stimulation against H1975 and A549 (FIGS. 3A-3C and 4A-4F). Control anti-ROR1 CAR T cells displayed minimal expansion in the presence of 10 ng/mL recombinant human TGFβ without exogenous IL2 through the first stimulation. Anti-ROR1 CAR T cells co-expressing TGFβR2-DNR, TGFβR2-DNR-CD2 and TGFβR2-DNR-OX40 exert enhanced cell proliferation and IFNg compared to anti-ROR1 CAR T cells alone. Anti-ROR1 CAR T cells co-expressing TGFβR2-DN and TGFβR2-DNR-CD2 show similar to increased IL2 production compared to Anti-ROR1 CAR T cells alone or coexpressing TGFβR2-DNR-CD28 (FIGS. 5A-5F). The sequential killing data also indicated anti-ROR1 CAR T cells co-expressing TGFβR2-DNR, TGFβR2-DNR-CD2 and TGFβR2-DNR-OX40 were able to sustain potent target lytic activities over several rounds of target exposure (FIGS. 6A-6C).

### Example 3—Overexpressing TGFbR2-DNR-CD2 in CAR T Cells Show Enhanced Proliferation and Sustained IL2 Production Against Target Cells

Co-stimulation of T cells with CD2 augments CD3-mediated signaling cascades, IL-2 production, and proliferation. Here, overexpressing TGFbR2-DNR chimeric receptor with CD2 intracellular domain switches the immunosuppressive TGFb signal to stimulatory signals.

Assay set-up—serial stimulation assay: Transduced primary T cells expressing R12 CAR with TGFβR2-DNR chimeric receptors were counted and resuspended to a cell density equal to 0.4×10⁶ CAR+ cells/ml in cell-assay media, and a 1 ml volume were added to a 12 well plate containing 0.4×10⁶ H1975-NLR cells in 1 ml cell-assay media and incubated at 37° C. +/− human recombinant TGFβ (10 ng/ml). No exogenous IL-2 was used for support in this assay. ROR1 CAR cells were counted and phenotyped every 7 days to measure cell proliferation and CAR %. ROR1 CAR cells coculture with ROR1⁺ target cells were reset in 1:1 E to T ratio every 7 days. Cytokine production (IFNγ and IL2) of ROR1 CAR cells was measured 24 hr after coculture set up each round by MSD.

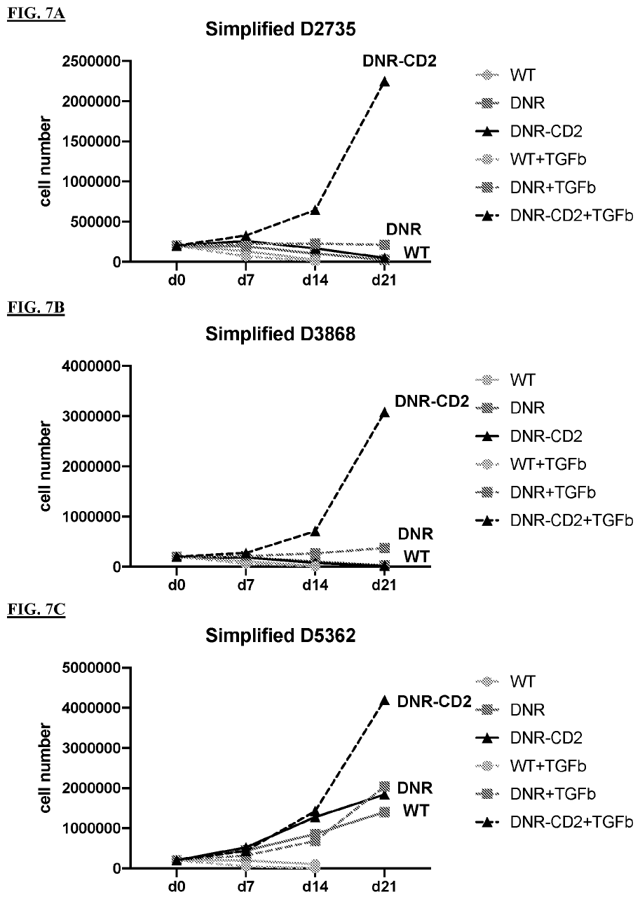

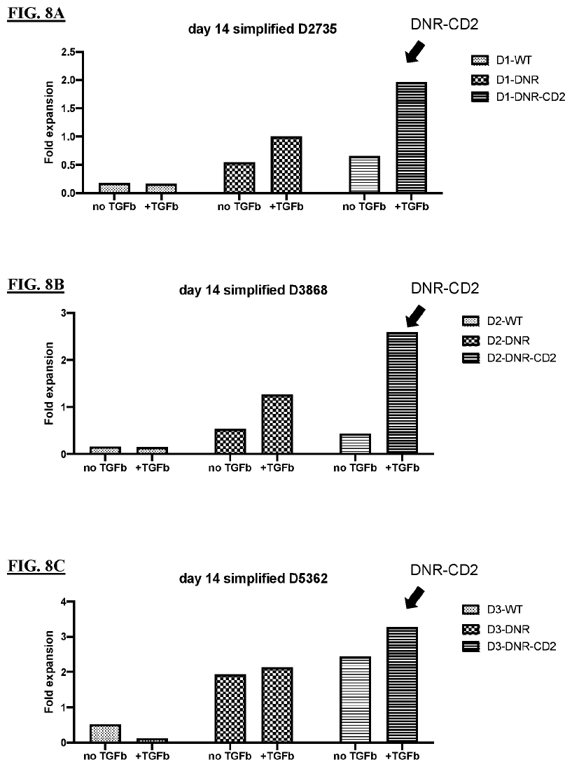

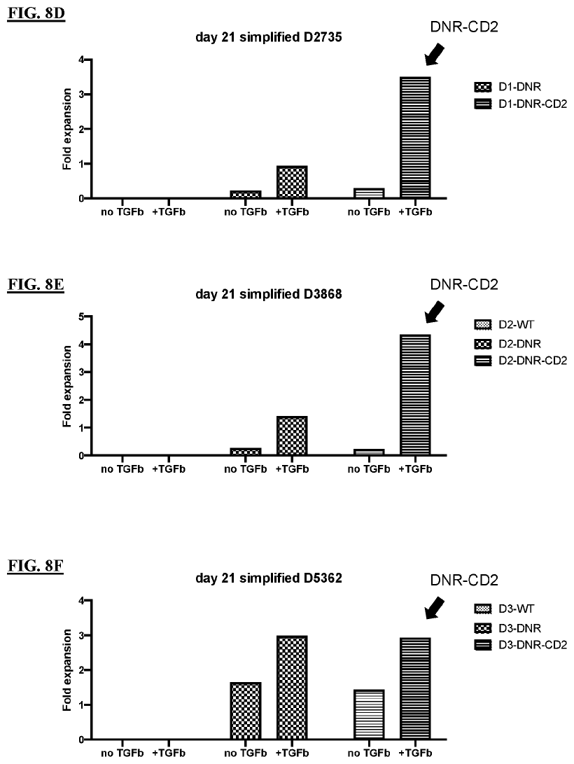

Result: In prolonged serial stimulation assay, control anti-ROR1 CAR T cells displayed minimal expansion in the presence of 10 ng/mL recombinant human TGFβ without exogenous IL2 through the first stimulation and were not cultured further. CAR T cells co-expressing the DNR also demonstrated reduced expansion when expanded in the presence of TGFβ in the later rounds. In contrast, anti-ROR1 CAR T cells co-expressing TGFβR2-DNR-CD2 were significantly protected from immunosuppressive TGFβ mediated signaling which increases T cell proliferation and reduces T cell hypofunction resulting from chronic antigen exposure. These data demonstrated that active CD2 signaling increased T cell expansion compared to the CAR alone. (FIGS. 7A-7C and 8A-8F)

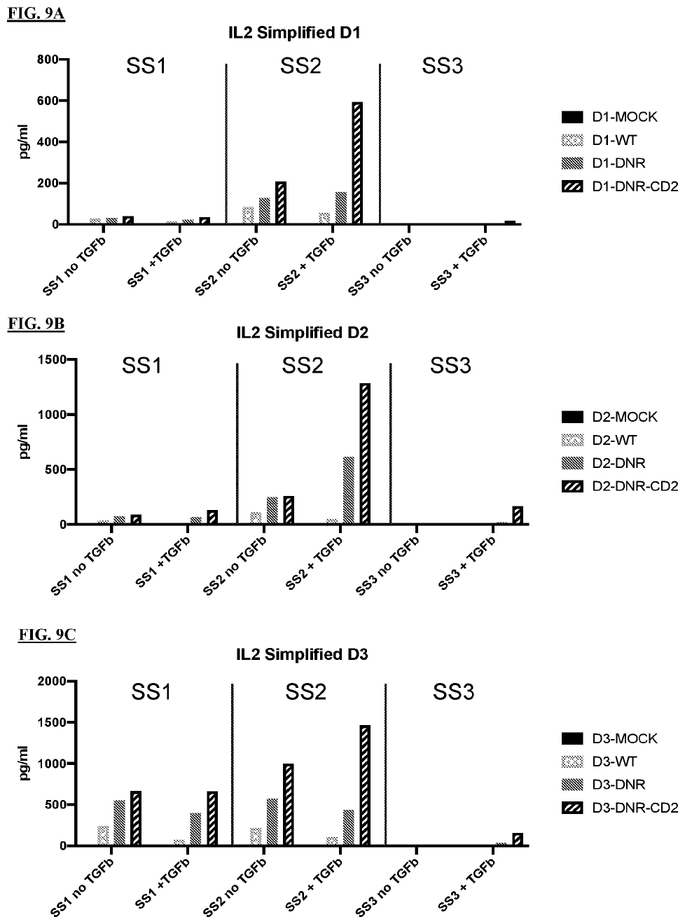

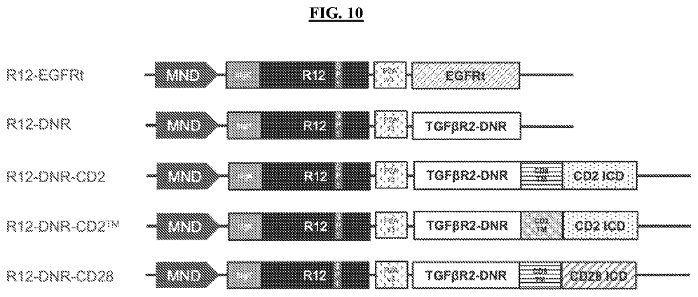

The results indicate TGFβR2-DNR-CD2 may show enhanced IL2 production in the presence of TGFβ at serial stimulation round 2 (day +8) and round 3 (day +15) comparing the DNR only. (FIGS. 9A-9C). Control anti-ROR1 CAR T cells displayed minimal IL2 production in the presence of 10 ng/mL recombinant human TGFβ. In contrast, anti-ROR1 CAR T cells co-expressing TGFβR2-DNR-CD2 were significantly protected from immunosuppressive TGFβ mediated signaling which increases IL2 production in the presence of TGF and chronic antigen exposure.

### Example 4— Overexpressing TGFbR2-DNR-CD2 with CD8 or CD2 Transmembrane in CAR T Cells Show Enhanced Proliferation and Sustained IL2 Production Against Target Cells as Compared to TGFbR2-DNR-CD28

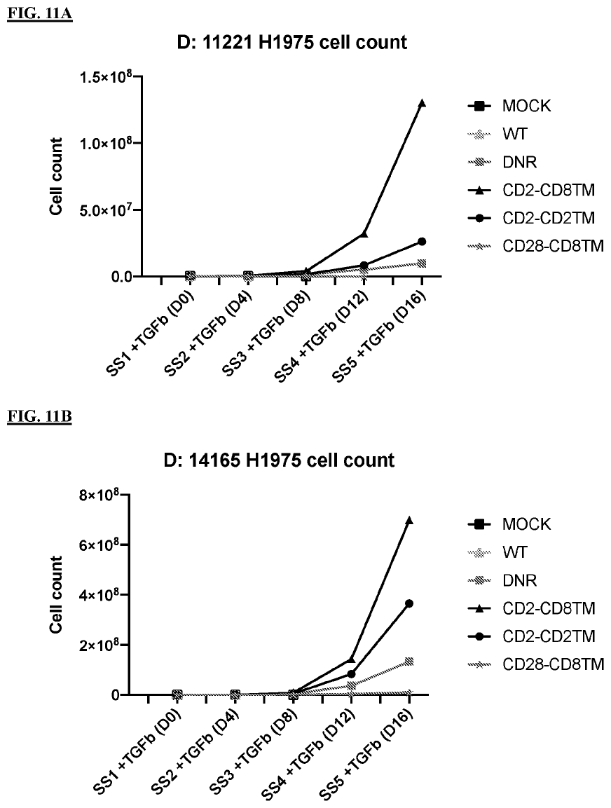

Illustrative TGFβR2-DNR chimeric receptor constructs were designed as shown in FIG. 10. Optimal T cell costimulatory signaling is initiated by the ligation to the corresponding ligands (CD2 and CD28). To convert immunosuppressive TGFβ signal to induce costimulatory signaling after exposure to TGFβ, the intracellular domain of TGFβR2 was replaced with T cell costimulatory signaling domains (CD2 and CD28). The TGFβR2 extracellular domain was connected to T cells costimulatory (CD2) signaling domains with a CD8a or endogenous CD2 transmembrane domain, CD28 costimulatory domains were connected to TGFβR2 extracellular domain with CD8a transmembrane domains respectively.

Assay set-up—serial stimulation assay: Transduced primary T cells expressing R12 CAR with TGFβR2-DNR chimeric receptors were counted and resuspended to a cell density equal to 0.4×10⁶ CAR+ cells/ml in cell-assay media, and a 1 ml volume were added to a 12-well plate containing 0.4×10⁶ H1975-NLR cells in 1 ml cell-assay media and incubated at 37° C. +/− human recombinant TGFβ (10 ng/ml). No exogenous IL-2 was used for support in this assay. ROR1 CAR cells were counted and phenotyped every 7 days to measure cell proliferation and CAR %. ROR1 CAR cells coculture with ROR1+ target cells were reset in 1:1 E to T ratio every 7 days. Cytokine production (IFNg and IL2) of ROR1 CAR cells was measured 24 hr after coculture set up each round by MSD.

Results:

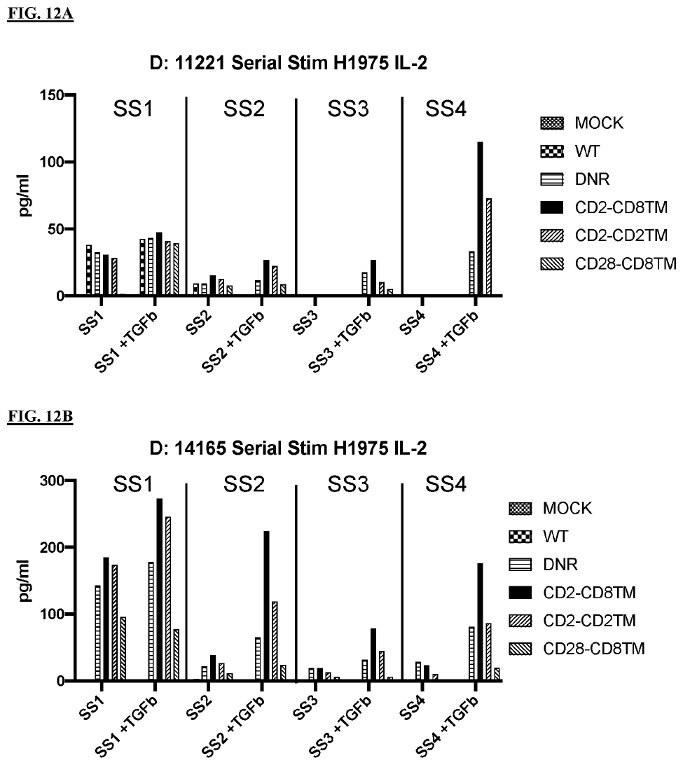

In prolonged serial stimulation assay, control anti-ROR1 CAR T cells displayed minimal expansion in the presence of 10 ng/mL recombinant human TGFβ without exogenous IL2 through the first stimulation and were not cultured further. CAR T cells co-expressing the control DNR also demonstrated reduced expansion when expanded in the presence of TGFβ in the later rounds. In contrast, anti-ROR1 CAR T cells co-expressing TGFβR2-DNR-CD2 were significantly protected from immunosuppressive TGFβ mediated signaling, thereby increasing T cell proliferation and reducing T cell hypofunction resulting from chronic antigen exposure. Surprisingly, anti-ROR1 CAR T cells co-expressing TGFβR2-DNR-CD2 were significantly protected from immunosuppressive TGFβ mediated signaling even as compared to anti-ROR1 CAR T cells co-expressing TGFβR2-DNR-CD28. These data demonstrated that active CD2 signaling increased T cell expansion compared to the CAR alone and as compared to the CAR T cells co-expressing TGFβR2-DNR, but without CD2 and indicate that active CD2 signaling provides a surprising benefit over CD28 signaling (FIGS. 11A-11B and 12A-12B).

The results also indicate anti-ROR1 CAR T co-expressing TGFβR2-DNR or TGFβR2-DNR-CD2 sustain CAR % throughout the serial stimulation assay. CAR % of control anti-ROR1 CAR T cells dropped after the first stimulation in the presence of 10 ng/mL recombinant human TGFβ.

The results indicate CAR T cells co-expressing TGFβR2-DNR-CD2 show enhanced IL2 production in the presence of TGFβ at serial stim round 2 (day +8) and round 3 (day +15) compared to CAR T cells co-expressing the DNR only. Control anti-ROR1 CAR T cells displayed minimal IL2 production in the presence of 10 ng/mL recombinant human TGFb. In contrast, anti-ROR1 CAR T cells co-expressing TGFβR2-DNR-CD2 were significantly protected from immunosuppressive TGFβ mediated signaling which increases IL2 production in the presence of TGFb and chronic antigen exposure.

### Example 5— Enhancement of Proliferation of TGFβR2-DNR-CD2 is Dependent on CAR Signaling

Assay set-up— target-independent serial stimulation assay: Transduced primary T cells expressing R12 CAR with TGFβR2-DNR chimeric receptors were counted and resuspended to a cell density equal to 0.4×10⁶ CAR+ cells/ml in cell-assay media, and a 1 ml volume were added to a 12 well plate containing 0.4×10⁶ A549-NLR cells or A549-NLR with ROR1-KO in 1 ml cell-assay media and incubated at 37° C. +/− human recombinant TGFβ (10 ng/ml). No exogenous IL-2 was used for support in this assay. ROR1 CAR cells were counted and phenotyped every 7 days to measure cell proliferation and CAR %. ROR1 CAR cells coculture with ROR1+ target cells were reset in 1:1 E to T ratio every 7 days.

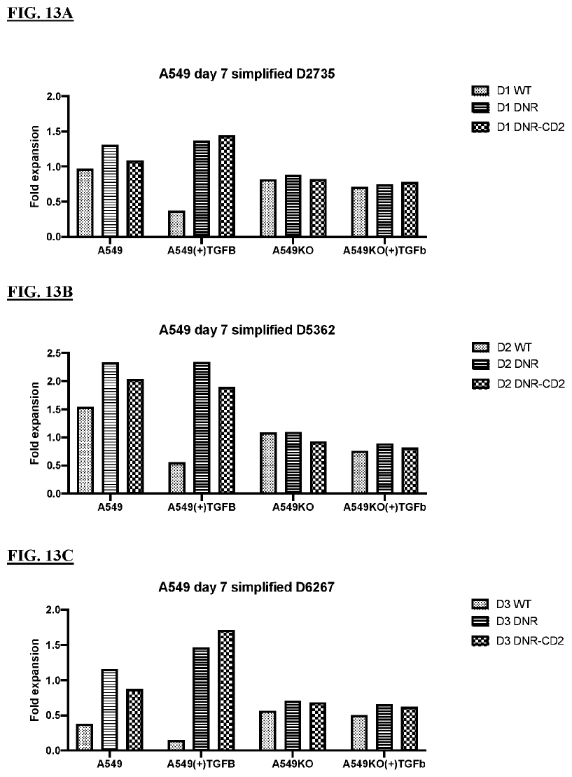

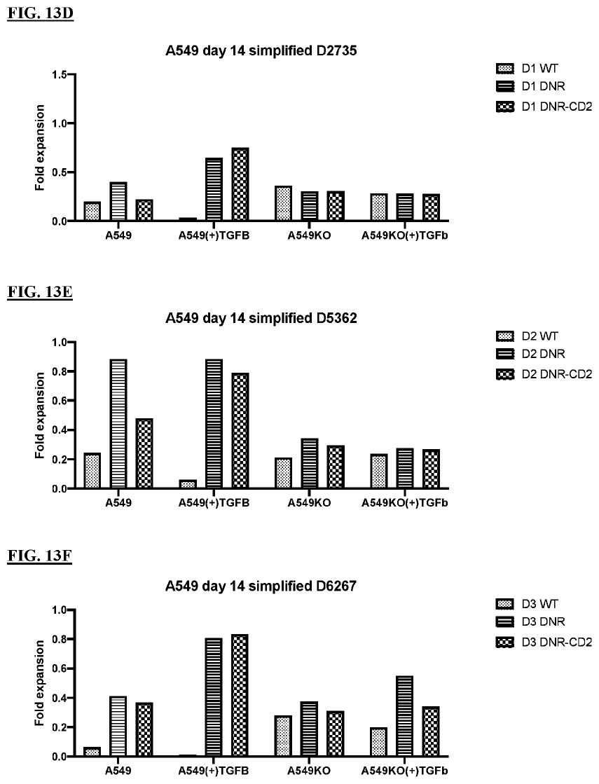

Results: TGFβ signaling decreases T cell expansion in response to antigen stimulation. Control anti-ROR1 CAR T cells displayed minimal expansion in the presence of 10 ng/mL recombinant human TGFβ without exogenous IL2. In contrast, anti-ROR1 CAR T cells co-expressing TGFβR2-DNR-CD2 were significantly protected from immunosuppressive TGFβ mediated signaling which increases T cell proliferation and reduces T cell hypofunction resulting from chronic antigen exposure. The TGFβ-induced enhancement of proliferation is CAR target dependent. No TGFβ-induced proliferation observed with A549-NLR ROR1-KO cells (FIGS. 13A-13F).

***

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

What is claimed is:

1. An immune cell comprising a chimeric activation receptor, wherein the chimeric activation receptor comprises (i) a transforming growth factor β (TGFβ)-binding domain; (ii) a transmembrane domain; (iii) and a CD2 costimulatory domain; wherein the immune cell expresses an endogenous TGFβRI and/or TGFβRII.

2. The immune cell of claim 1, which is selected from a T cell, a B cell, a regulatory T cell (Treg), a natural killer (NK) cell, a natural killer T (NKT) cell, a stem cell, an induced pluripotent stem cell, and any combination thereof.

3. The immune cell of claim 1 or 2, wherein the chimeric activation receptor is capable of competing with binding of an endogenous TGFβRI and/or an endogenous TGFβRII to TGFb.

4. The immune cell of any one of claims 1 to 3, wherein the chimeric activation receptor is capable of forming a heterotetradimer with an endogenous TGFβRI and/or an endogenous TGFβRII.

5. The immune cell of any one of claims 1 to 4, wherein the TGFβ-binding domain is an extracellular domain of TGFβRII.

6. The immune cell of any one of claims 1 to 5, wherein upon interaction of the chimeric activation receptor with TGFβ, the immune cell produces one or more cytokines at a higher level than a cell expressing a TGFβRII-binding domain fused to a CD28 costimulatory domain upon interaction with TGFβ.

7. The immune cell of claim 6, wherein the one or more cytokines are selected from IL2 and IFNγ.

8. The immune cell of any one of claims 1 to 7, wherein upon the interaction with TGFβ, the immune cell expresses IL2 at a level that is at least about 125%, at least about 150%, at least about 200%, at least about 250%, at least about 300%, at least about 350%, at least about 400%, at least about 450%, at least about 500% the expression of IL2 by a cell expressing a TGFβRII-binding domain fused to a CD28 costimulatory domain upon binding to TGFβ.

9. The immune cell of any one of claims 1 to 8, wherein upon the interaction with TGFβ, the immune cell expresses IFNγ at a level that is at least about 125%, at least about 150%, at least about 200%, at least about 250%, at least about 300%, at least about 350%, at least about 400%, at least about 450%, at least about 500% the expression of IFNγ by a cell expressing a TGFβRII-binding domain fused to a CD28 costimulatory domain upon binding to TGFβ.

10. The immune cell of any one of claims 6 to 9, wherein the expression of the one or more cytokines by the immune cell is higher than a cell expressing a TGFβRII-binding domain fused to a CD28 costimulatory domain, as measured at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

11. The immune cell of any one of claims 1 to 10, wherein upon the interaction with TGFβ, the immune cell is more proliferative than a cell comprising a TGFβRII-binding domain fused to a CD28 costimulatory domain upon interaction with TGFβ.

12. The immune cell of any one of claims 1 to 11, wherein upon interaction with TGFβ, the immune cell is at least about 25% more, at least about 50% more, at least about 75% more, at least about 100% more, at least about 125% more, at least about 150% more, at least about 175% more, at least about 200% more, at least about 250% more, at least about 300% more, at least about 350% more, at least about 400% more, at least about 450% more, at least about 500% more proliferative than a cell comprising a TGFβRII-binding domain fused to a CD28 costimulatory domain upon interaction with TGFβ.

13. The immune cell of any one of claims 1 to 12, wherein upon interaction with TGFβ, the immune cell is more proliferative than a cell comprising a TGFβRII-binding domain fused to a CD28 costimulatory domain, as measured at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

14. The immune cell of any one of claims 1 to 13, wherein upon binding to TGFβ, the immune cell has increased cytolytic activity as compared to a cell comprising a TGFβRII-binding domain fused to a CD28 costimulatory domain upon binding to TGFβ.

15. The immune cell of any one of claims 1 to 14, wherein upon interaction with TGFβ, the immune cell has a cytolytic activity that is at least about 125%, at least about 150%, at least about 200%, at least about 250%, at least about 300%, at least about 350%, at least about 400%, at least about 450%, at least about 500% the cytolytic activity of a cell comprising a TGFβRII-binding domain fused to a CD28 costimulatory domain upon interaction with TGFβ.

16. The immune cell of any one of claims 1 to 15, wherein upon interaction with TGFβ, the immune cell has increased cytolytic activity as compared to a cell comprising a TGFβRII-binding domain fused to a CD28 costimulatory domain, as measured at least about 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, or at least 14 days after a first interaction with TGFβ.

17. The immune cell of any one of claims 1 to 16, wherein the TGFβ-binding domain comprises the extracellular domain of wild-type human TGFβRII.

18. The immune cell of any one of claims 1 to 17, wherein the TGFβ-binding domain comprises the extracellular domain of human TGFβRII having one or more point mutation relative to wild-type human TGFβRII, which increases the binding affinity of the extracellular domain of TGFβRII to TGFβ.

19. The immune cell of any one of claims 1 to 18, wherein the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6.

20. The immune cell of any one of claims 1 to 19, wherein the TGFβ-binding domain comprises the amino acid sequence set forth in SEQ ID NO: 6.

21. The immune cell of any one of claims 1 to 20, wherein the transmembrane domain comprises the transmembrane domain selected from the group consisting of wild-type human TGFβRII, a CD8 transmembrane domain, a CD2 transmembrane domain, and any combination thereof.

22. The immune cell of any one of claims 1 to 20, wherein the transmembrane domain comprises a CD8 transmembrane domain.

23. The immune cell of any one of claims 1 to 22, wherein the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5, 8, or 9.

24. The immune cell of any one of claims 1 to 23, wherein the transmembrane domain comprises the amino acid sequence set forth in SEQ ID NO: 5, 8, or 9.

25. The immune cell of any one of claims 1 to 24, wherein the CD2 costimulatory domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7.

26. The immune cell of any one of claims 1 to 25, wherein the CD2 costimulatory domain of comprises the amino acid sequence set forth in SEQ ID NO: 7.

27. The immune cell of any one of claims 1 to 26, further comprising a chimeric antigen receptor and/or a TCR.

28. The immune cell of claim 27, wherein the chimeric antigen receptor comprises an antigen-binding domain that specifically binds a molecule expressed by a tumor cell.

29. The immune cell of claim 27 or 28, wherein the chimeric antigen receptor comprises an antigen-binding domain that specifically binds an antigen selected from the group consisting of AFP (alpha-fetoprotein), αvβ6 or another integrin, BCMA, Braf, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof.

30. The immune cell of claim 29, wherein the chimeric antigen receptor comprises an antigen-binding domain that specifically binds ROR1.

31. The immune cell of claim 29, wherein the chimeric antigen receptor comprises an antigen-binding domain that specifically binds GPC2.

32. The immune cell of any one of claims 27 to 31, wherein the chimeric antigen receptor comprises a costimulatory domain selected from a costimulatory domain from interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, and any combination thereof.

33. The immune cell of any one of claims 27 to 32, wherein the chimeric antigen receptor comprises a 4-1BB/CD137 costimulatory domain.

34. The immune cell of any one of claims 27 to 33, wherein the TCR specifically binds a tumor antigen.

35. The immune cell of any one of claims 27 to 34, wherein the TCR specifically binds an antigen selected from the group consisting of AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

36. A nucleic acid encoding the chimeric activation receptor of any one of claims 1 to 35.

37. A nucleic acid encoding a chimeric activation receptor comprising (i) a transforming growth factor (3 (TGFβ)-binding domain; (ii) a transmembrane domain; (iii) and a CD2 costimulatory domain.

38. The nucleic acid of claim 37, wherein the TGFβ-binding domain is an extracellular domain of TGFβRII.

39. The nucleic acid of claim 37 or 38, wherein the TGFβ-binding domain comprises the extracellular domain of wild-type human TGFβRII.

40. The nucleic acid of any one of claims 37 to 39, wherein the TGFβ-binding domain comprises the extracellular domain of human TGFβRII having one or more point mutation relative to wild-type human TGFβRII, which increases the binding affinity of the extracellular domain of TGFβRII to TGFβ.

41. The nucleic acid of any one of claims 37 to 40, wherein the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6.

42. The nucleic acid of any one of claims 37 to 41, wherein the TGFβ-binding domain comprises the amino acid sequence set forth in SEQ ID NO: 6.

43. The nucleic acid of any one of claims 37 to 42, wherein the transmembrane domain comprises the transmembrane domain of wild-type human TGFβRII.

44. The nucleic acid of any one of claims 37 to 43, wherein the transmembrane domain comprises a CD8 transmembrane domain.

45. The nucleic acid any of one of claims 37 to 44, wherein the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5, 8, or 9.

46. The nucleic acid of any one of claims 37 to 45, wherein the transmembrane domain comprises the amino acid sequence set forth in SEQ ID NO: 5, 8 or 9.

47. The nucleic acid of any one of claims 37 to 46, wherein the CD2 costimulatory domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7.

48. The nucleic acid of any one of claims 37 to 47, wherein the CD2 costimulatory domain of comprises the amino acid sequence set forth in SEQ ID NO: 7.

49. The nucleic acid of any one of claims 37 to 48, further encoding a chimeric antigen receptor.

50. The nucleic acid of claim 49, wherein the chimeric antigen receptor comprises an antigen-binding moiety that specifically binds a target molecule expressed by a tumor cell.

51. The nucleic acid of claim 50, wherein the antigen-binding moiety comprises a fragment of an antibody.

52. The nucleic acid of any of claim 50 or 51, wherein the antigen-binding moiety comprises an scFv, a nanobody, a VHH, an Fab, a DARPin, a vNAR, or an affibody.

53. The nucleic acid of any one of claims 50 to 52, wherein the antigen-binding moiety specifically binds an antigen selected from the group consisting of AFP (alpha-fetoprotein), αvβ6 or another integrin, BCMA, Braf, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof.

54. The nucleic acid of any one of claims 50 to 53, wherein the antigen-binding moiety specifically binds GPC2.

55. The nucleic acid of any one of claims 50 to 53, wherein the antigen-binding moiety specifically binds ROR1.

56. The nucleic acid of any one of claims 49 to 55, further encoding a linker between the chimeric antigen receptor and the chimeric signaling receptor.

57. The nucleic acid of any one of claims 37 to 48, further encoding a TCR.

58. The nucleic acid of claim 57, wherein the TCR specifically binds a tumor antigen.

59. The nucleic acid of claim 57 or 58, wherein the TCR specifically binds an antigen selected from the group consisting of AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin Bl, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

60. The nucleic acid of any one of claims 57 to 59, further encoding a linker between the TCR and the chimeric signaling receptor.

61. The nucleic acid of claim 56 or 60, wherein the linker is a cleavable linker.

62. The nucleic acid of claim 61, wherein the linker is selected from a P2A linker, a T2A linker, an F2A linker, an E2A linker, a furin cleavage site, or any combination thereof.

63. The nucleic acid of any one of claims 56 and 60 to 62, wherein the linker comprises an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 11.

64. The nucleic acid of any one of claims 56 and 60 to 63, wherein linker comprises the amino acid sequence set forth in SEQ ID NO: 11.

65. The nucleic acid of any one of claims 50 to 64, further comprising an IRES in between the portion of the nucleic acid encoding the chimeric antigen receptor and the portion of the nucleic acid encoding the chimeric activation receptor.

66. An expression vector comprising the nucleic acid of any one of claims 36 to 65 operably linked to a regulatory sequence.

67. The expression vector of claim 66, which is a lentiviral vector, a retroviral vector, a bacterial vector, a DNA plasmid, a dsDNA fragment, an ssDNA fragment, or any combination thereof.

68. A chimeric activation receptor comprising (i) a transforming growth factor β (TGFβ)-binding domain; (ii) a transmembrane domain; (iii) and a CD2 costimulatory domain.

69. The chimeric activation receptor claim 68, wherein the TGFβ-binding domain is an extracellular domain of TGFβRII.

70. The chimeric activation receptor of claim 68 or 69, wherein the TGFβ-binding domain comprises the extracellular domain of wild-type human TGFβRII.

71. The chimeric activation receptor of any one of claims 68 to 70, wherein the TGFβ-binding domain comprises the extracellular domain of human TGFβRII having one or more point mutation relative to wild-type human TGFβRII, which increases the binding affinity of the extracellular domain of TGFβRII to TGFβ.

72. The chimeric activation receptor of any one of claims 68 to 71, wherein the TGFβ-binding domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 6.

73. The chimeric activation receptor of any one of claims 68 to 72, wherein the TGFβ-binding domain comprises the amino acid sequence set forth in SEQ ID NO: 6.

74. The chimeric activation receptor of any one of claims 68 to 73, wherein the transmembrane domain comprises the transmembrane domain of wild-type human TGFβRII, CD2, CD8, or any combination thereof.

75. The chimeric activation receptor of any one of claims 68 to 74, wherein the transmembrane domain comprises a CD8 transmembrane domain.

76. The chimeric activation receptor any of one of claims 68 to 74, wherein the transmembrane domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 5, 8, or 9.

77. The chimeric activation receptor of any one of claims 68 to 76 wherein the transmembrane domain comprises the amino acid sequence set forth in SEQ ID NO: 5, 8, or 9.

78. The chimeric activation receptor of any one of claims 68 to 77, wherein the CD2 costimulatory domain comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7.

79. The chimeric activation receptor of any one of claims 68 to 78, wherein the CD2 costimulatory domain comprises the amino acid sequence set forth in SEQ ID NO: 7.

80. A chimeric activation receptor, encoded by the nucleic acid of any one of claims 37 to 65.

81. A pharmaceutical composition comprising the immune cell of any one of claims 1 to 35, the nucleic acid of any one of claims 36 to 65, the vector of claim 66 or 67, or the chimeric activation receptor of any one of claims 68 to 80.

82. A method of preparing a cell expressing a chimeric activation receptor comprising transfecting a cell with the nucleic acid of any one of claims 36 to 65.

83. A method of converting an endogenous TGFβR activity to a stimulatory signaling in a cell comprising transfecting the immune cell with the nucleic acid of any one of claims 36 to 65.

84. A method of modulating TGFβ activity in a tumor microenvironment comprising administering the immune cell of any one of claims 1 to 35.

85. A method of treating a tumor in a subject in need thereof, comprising administering to the subject the immune cell of any one of claims 1 to 35.

86. The method of claim 85, wherein the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

87. The method of claim 85 or 86, wherein the tumor is a solid tumor.

88. The method of any one of claims 85 to 87, wherein the tumor microenvironment comprise one or more cells that express TGFβ.

89. The method of any one of claims 85 to 88, wherein a tumor cell expresses TGFβ.

90. The method of any one of claims 85 to 89, wherein one or more fibroblasts, MDSC-myeloid derived suppressor cells, Treg, macrophages, or any combination thereof in the tumor microenvironment express TGFβ.


SEQUENCE LISTING (21 sequences)

SEQ ID NO: 1 - protein, 503 aa, Artificial Sequence
  Human TGF(beta)RI (signal peptide; extra- cellular domain; trans-membrane domain)
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 2 - protein, 21 aa, Artificial Sequence
  Human TGF(beta)RI trans-membrane domain
AAVIAGPVCFVCISLMLMVYI

SEQ ID NO: 3 - protein, 93 aa, Artificial Sequence
  Human TGF(beta)RI extra-cellular domain
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 4 - protein, 567 aa, Artificial Sequence
  Human TGF(beta)RII (signal peptide; extra- cellular domain; trans-membrane domain)
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 5 - protein, 21 aa, Artificial Sequence
  Human TGF(beta)RII trans-membrane domain
VTGISLLPPLGVAISVIIIFY

SEQ ID NO: 6 - protein, 144 aa, Artificial Sequence
  Human TGF(beta)RII extra-cellular domain
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 7 - protein, 116 aa, Artificial Sequence
  CD2 Costimulatory Domain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 8 - protein, 24 aa, Artificial Sequence
  CD8 TM Domain
IYIWAPLAGTCGVLLLSLVITLYC

SEQ ID NO: 9 - protein, 26 aa, Artificial Sequence
  CD2 TM Domain
IYLIIGICGGGSLLMVFVALLVFYIT

SEQ ID NO: 10 - skipped

SEQ ID NO: 11 - protein, 19 aa, Artificial Sequence
  P2A
ATNFSLLKQAGDVEENPGP

SEQ ID NO: 12 - protein, 18 aa, Artificial Sequence
  T2A
EGRGSLLTCGDVEENPGP

SEQ ID NO: 13 - protein, 22 aa, Artificial Sequence
  F2A
VKQTLNFDLLKLAGDVESNPGP

SEQ ID NO: 14 - protein, 20 aa, Artificial Sequence
  E2A
QCTNYALLKLAGDVESNPGP

SEQ ID NO: 15 - protein, 4 aa, Artificial Sequence
  Furin Cleavage Site
RAKR

SEQ ID NO: 16 - protein, 41 aa, Artificial Sequence
  CD28 costimulatory domain
RSKRSRLLHSDYMNMTPRRPGPTRKHYQPYAPPRDFAAYRS

SEQ ID NO: 17 - protein, 63 aa, Artificial Sequence
  OX40 costimulatory domain
VAAILGLGLVLGLLGPLAILLALYLLRRDQRLPPDAHKPPGGGSFRTPIQEEQADAHSTLAKI

SEQ ID NO: 18 - protein, 93 aa, Artificial Sequence; identical to SEQ ID NO: 3
  Synthetic sequence

SEQ ID NO: 19 - protein, 144 aa, Artificial Sequence; identical to SEQ ID NO: 6
  Synthetic sequence

SEQ ID NO: 20 - protein, 39 aa, Artificial Sequence
  CD28 TM Domain
CPSPLFPGPSKPFWVLVVVGGVLACYSLLVTVAFIIFWV

SEQ ID NO: 21 - protein, 21 aa, Artificial Sequence
  OX40 TM Domain
VAAILGLGLVLGLLGPLAILL